(19) United States
(12) Patent Application Publication
(10) Pub. No.: US 2022/0068478 A1
(43) Pub. Date: Mar. 3, 2022

(54) SCALABLE, ACCURATE AND RELIABLE MEASURE OF VARIABLE DEPENDENCE AND INDEPENDENCE, AND UTILIZATION OF THE MEASURE TO TRAIN A NEURAL NETWORK

(71) Applicant: NEC Laboratories Europe GmbH, Heidelberg (DE)

(72) Inventors: Shujian Yu, Heidelberg (DE); Francesco Alesiani, Heidelberg (DE)

(21) Appl. No.: 17/161,728

(22) Filed: Jan. 29, 2021

(30) Foreign Application Priority Data
Sep. 1, 2020 (EP) 20193887

(51) Int. Cl. G16H 50/20; G16H 20/00; G06N 3/08

(52) U.S. Cl. CPC G16H 50/20; G06N 3/08; G16H 20/00

(57) ABSTRACT

A method for measuring dependence and independence of variables includes collecting data for the variables, computing sample Gram matrices for the variables, and computing element-wise products of the sample Gram matrices. Normalized mutual information of the variables is then computed using Renyi's α-order entropy function based on the element-wise products as an independence measure of the variables.

### CROSS-REFERENCE TO RELATED APPLICATION

Priority is claimed to European Patent Application No. EP 20193887, filed on Sep. 1, 2020, the entire disclosure of which is hereby incorporated by reference herein.

### FIELD

The present invention relates to a method, system and computer-readable medium for measuring the degree of dependence of variables on one another, with applications in machine learning, and to the training of a neural network based on the measure.

### BACKGROUND

Measuring the degree of dependence among multiple random variables, which may be vectors, plays a central role in machine learning, information theory, and statistics. Measurements of the dependence of variables has applications in a number of technical fields such as bioinformatics, blind source separation, computer vision (e.g., image registration), causal analysis, automated finance, automated clinical evaluation, soil surveying, noise cancellation, speaker separation and identification, face recognition, object detection, feature selection in machine learning, independent component analysis, etc.

For example, given the quantities X1, . . . , Xd that represent the occurrence of some event or the level of some variables, there exists the technical problem to determine, for example, whether the occurrence of two events is independent, or if these events are dependent to a common ancestor, or if there is a third element that connects directly the two quantities. If the variables are modeled as random variables defined by some distribution, it is possible to measure the independence using existing tools. However, such existing tools are not scalable and are inaccurate and unreliable, among other shortcomings. For example, a simple statistical correlation measure such as the Pearson correlation does not capture higher order information from the distribution of the variables.

The mutual information is defined as:

I(X,Y)=H(X)+H(Y)−H(X,Y)

The common approach is to evaluate these quantities using the empirical distribution (i.e., the distribution derived from the samples). However, the general computation of the probability distribution or some of its moments is noisy and affected by the curse of dimensionality. This means that the dimension of the variable increases too high, preventing scalability, and the empirical distribution is too noisy, preventing accuracy and reliability, such that the approach cannot be effectively used in a number of practical applications.

For a linear dependence case, measures such as the Pearson's rho, the Spearman's rank and the Kendall's tau are computationally efficient and have been widely used. For the more general case where the two variables share a non-linear relationship, one of the most well-known dependence measures is the mutual information and its modifications (e.g., the Cauchy-Schwartz quadratic mutual information and the randomized information coefficient). However, these measures are limited to measuring dependencies between scalar random variables (e.g., the maximal information coefficient) and/or are difficult to be interpret. For example, most kernel independence measures have a zero expected value if and only if the associated random variables are independent, given universal kernels. However, when the empirical estimates of these measures are not zero, it is hard to tell if the value indicates a statistically significant dependence. Moreover, the utilities of these methods on more practical machine learning applications, beyond the scope of a sample estimator to the degree of dependence, are largely unknown. For example, it is not known whether it is possible to design neural networks based on those measures, or how that could be achieved.

### SUMMARY

In an embodiment, the present invention provides a method for measuring dependence and independence of variables. The method includes collecting data for the variables, computing sample Gram matrices for the variables, computing element-wise products of the sample Gram matrices, and computing normalized mutual information of the variables using Renyi's α-order entropy function based on the element-wise products as an independence measure of the variables.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a method for computation of the normalized mutual information to test variable dependence/independence and determine a dependence/independence measure according to an embodiment of the present invention;

FIG. 2 schematically illustrates an embodiment of the present invention in which the dependence/independence measure is a loss function in training a neural network;

FIG. 3 schematically illustrates a method for training a neural network in accordance with an embodiment of the present invention;

FIG. 4 illustrates gene expression;

FIG. 5 schematically illustrates a method and system for automatic gene regulatory network (GRN) generation according to an embodiment of the present invention;

FIG. 6 schematically illustrates application of the independence measure/test and GRN inference in accordance with an embodiment of the present invention;

FIG. 7 schematically illustrates an embodiment of the present invention for informative gene set selection using the independence measure/test;

FIG. 8 schematically illustrates an embodiment of the present invention applicable to the technical problem of blind source separation with multiple speakers;

FIG. 9 schematically illustrates an embodiment of the present invention applicable to the technical problem of blind source separation in petroleum exploration;

FIG. 10 is a graph quantitatively demonstrating increased accuracy possible with embodiments of the present invention compared to other approaches;

FIGS. 11a and 11b are graphs comparing performance of the independence measure according to embodiments of the present invention compared to other approaches;

FIGS. 12a and 12b show source images and target images, respectively, of the MNIST dataset used for one experiment;

FIGS. 13a and 13b show source images and target images, respectively, of the fashion MNIST dataset used for another experiment; and

FIGS. 14a, 14b, 14c and 14d show results experiments comparing the independence measure according to embodiments of the present invention (Renyi, indicated by stars) against different methods to compute correlation or independence.

### DETAILED DESCRIPTION

Embodiments of the present invention provide an accurate, reliable and scalable measure of variable dependence and independence. Determining such a measure is a critical task for many machine learning problems. Embodiments of the present invention are data driven and based on information theory to measure variable dependence and independence. Advantageously, the measure according to embodiments of the present invention is automatically differentiable. A version of the measure is normalized such that the measure is comparable across large number of conditions. The measure improves machine learning problems which use such a measure by increasing accuracy and reliability, and by being scalable. Additionally, the measure can be used to achieve improvements in a number of concrete technical applications such as the gene regularity network inference, informative gene selection, speaker separation, geological exploration and petroleum exploration, among many others.

Embodiments of the present invention provide a way to avoid the computation of the empirical distributions and still have the same accuracy with the closed form of the distributions.

Robust machine learning under domain shift or non-Gaussian noises has attracted increasing attention in recent years. This is because the success of deep learning models is highly dependent on the assumption that the training and testing data are independent and identically distributed and sampled from the same distribution (with low or moderate levels of Gaussian noises). Unfortunately, the data in reality is typically collected from different, but related domains, and is contaminated with non-Gaussian noises. In an embodiment, the present invention provides that the measure of the dependence/independence of the variables is utilized as a loss function that is robust to both domain shift as well as non-Gaussian noises, especially when there is no access to the samples from the target domain.

As exemplary embodiments, the following discussion describes how to compute a measure of the dependence/independence of variables (which can also be referred to as the dependence measure or, as used most often herein, as the independence measure) and how this measure is used for:
1) Gene regulatory network inference and automatic experiment design.
2) Informative gene selection.
3) Blind source separation in signal processing for source separation, speaker separation (identification) and soil/petroleum exploration.

In gene regulatory network inference, accurately separating an actual active gene from noisy or unrelated factors provides a number of improvements. If the gene regulatory network contains errors in description, this leads to the necessity of more experiments or to development of chemical elements that are not effective. In bioinformatics, there are a number of applications for an independence measure, for example, to determine which genes among thousands of genes are statistically relevant to the prediction of patient's heart disease, as well as to determine how they are associated with the heart disease. In petroleum exploration or source separation, providing a low level of uncertainty advantageously avoids costly exploration activities which have high social and geological impacts, or avoids misidentification or provides for clear signal recovery.

The foregoing exemplary applications involve the precise identification of the interactions (typically the dependence and the independence) among multiple (usually more than three) variables. In some cases, each variable is in a high-dimensional space, rather than a simple scalar.

In an embodiment, the present invention provides a method for measuring dependence and independence of variables. The method includes collecting data for the variables, computing sample Gram matrices for the variables, computing element-wise products (e.g., Hadamard products) of the sample Gram matrices, and computing normalized mutual information of the variables using Renyi's α-order entropy function based on the element-wise products as an independence measure of the variable, in particular in a way that provides differentiability and interpretability.

In an embodiment, the independence measure is automatically differentiable.

In an embodiment, the method further comprises utilizing the independence measure as a loss function for training a neural network.

In an embodiment, the variables are gene expressions, the method further comprising utilizing the independence measure to build a gene regulatory network (GRN) in which nodes represent levels of the gene expressions and edges represent interactions between genes.

In an embodiment, the method further comprises utilizing the GRN to design a compound, classify biological samples, design a treatment or to identify a most informative set of genes for a particular disease.

In an embodiment, the variables are signals, the method further comprising utilizing the independence measure for blind source separation to distinguish different sources of the signals.

In an embodiment, the blind source separation is used to distinguish between and listen to multiple speakers, or to measure propagation delay of seismic waves for petroleum exploration.

In an embodiment, the independence measure between variable is between [0,1] with 0 indicating complete independence and 1 indicating direct functional dependence.

In an embodiment, a normalization factor used to obtain the normalized mutual information is based on an upper bound of the mutual information using Shearer's inequality.

In another embodiment, the present invention provides a system for measuring dependence and independence of variables comprising memory and one or more hardware processors which, alone or in combination, are configured to provide for execution of the following steps: collecting data for the variables; computing sample Gram matrices for the variables; computing Hadamard products of the sample Gram matrices; and computing normalized mutual information of the variables using Renyi's α-order entropy function based on the Hadamard products as an independence measure of the variables.

In an embodiment, the independence measure is automatically differentiable, the system being further configured to utilize the independence measure as a loss function for training a neural network.

In an embodiment, the variables are gene expressions, the system being further configured to utilize the independence measure to build a gene regulatory network (GRN) in which nodes represent levels of the gene expressions and edges represent interactions between genes, the GRN being useable to design a compound, classify biological samples, design a treatment or to identify a most informative set of genes for a particular disease.

In an embodiment, the variables are signals, the system being further configured to utilize the independence measure for blind source separation to distinguish different sources of the source signals in order to distinguish between and listen to multiple speakers, or to measure propagation delay of seismic waves for petroleum exploration.

In an embodiment, the independence measure between variables is between [0,1] with 0 indicating complete independence and 1 indicating direct functional dependence.

In a further embodiment, the present invention provides a tangible, non-transitory computer-readable medium having instructions thereon which, upon being executed by one or more processors, alone or in combination, provide for execution of any of the methods according to an embodiment of the present invention.

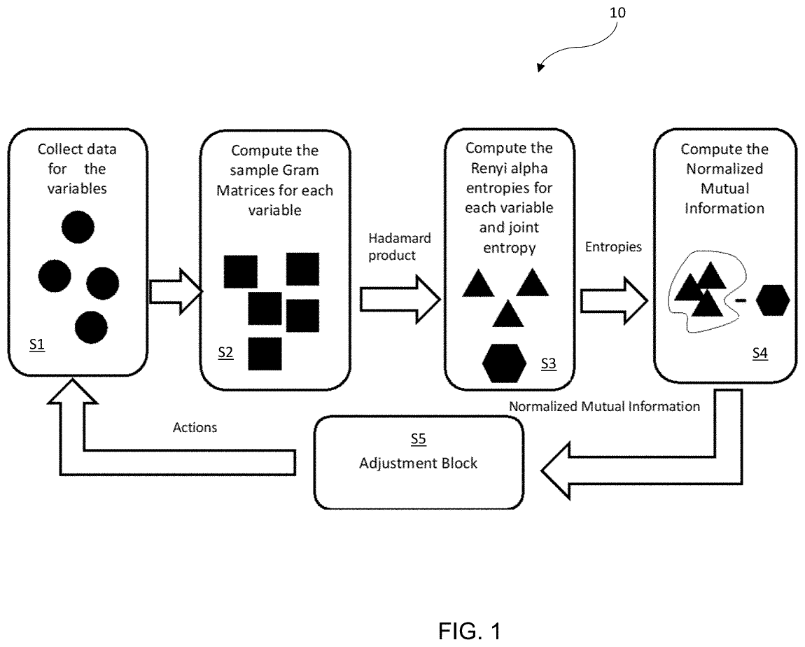

FIG. 1 schematically shows the steps of a method 10 for determining the dependence/independence measure of variable in accordance with an embodiment of the present invention. The method comprises the following steps:
S1) Collect the data for each of the variables;
S2) Compute the Gram matrices for each variable and each sample;
S3) Compute the Renyi alpha entropies based on the Gram matrices for the single variables and the joint distribution;
S4) Compute the normalization factor, and the mutual information, and compute the normalized mutual information as the dependence/independence measure; and
S5) In some situations (e.g., when working in a system that is evaluated or affected by the measure), adapt the variables involved in the process (e.g., in the training of a neural network, the aim is to minimize the dependence between input and error, so it is advantageous to evaluate the dependence in each step of the training process, and use the measure to adjust the weights of a neural network).

There is one Gram matrix per variable, which matrix has a size proportional to the number of samples (N times N, if N is the number of samples) and in this regard can be referred to as a sample Gram matrix. The sample Gram matrix can also be computed based on a kernel function, so it can also be referred to as a kernel Gram matrix or a sample kernel Gram matrix. If there are L variables, then there are L sample kernel Gram matrices.

For performing steps S2 and S3, the following procedure can be used:
1) Give data X={xᵢₗ},
   a. i=1, . . . , N (samples)
   b. L=1, . . . , L (variables)
2) Given the kernel function k(x,y)
3) Build L matrices Gₗ
   a. l=1, . . . , L
   b. Gₗ={g_{{ij,l}}}
   c. g_{{ij,l}}=k(xᵢₗ,xⱼₗ) represent a kernel function as the entry of the matrix used to compute the entropy of the variable, wherein K is the entry correlation of samples of the variable.
4) Build the Hadamard product
   a. H=G₁ o . . . o G_{L}
5) Compute the Total Correlations T*,D* based on the equations and the previous matrices based on the Renyi alpha entropies (Sα)

For computing Renyi alpha entropies, the following procedure can be used:
1) Sα(A)=1/(1−α) log tr A^{α} (single entropy)
2) Sα(A₁, . . . , A_{L})=Sα(A₁ o . . . o A_{L}/tr (A₁ o . . . o A_{L})) (joint entropy)

In step S4 of FIG. 1, the normalization factor is computed based on the Shearer's inequality which is the computation of the entropy when one (or more) variable is excluded (the average of all possible subset of variables of a specified size (e.g., 1). In FIG. 1, let us look at the right most block. Each triangle in step S4 of FIG. 1 denotes the entropy for one variable. The entropy from all individual variables are summed (which is represented by a circle around all triangles). The hexagon in step S4 of FIG. 1 then denotes the joint entropy from all variables when they are available simultaneously. The sum of entropy from each variable is compared against the joint entropy from all variables (denoted by comparing the combination of all triangles with the hexagon).

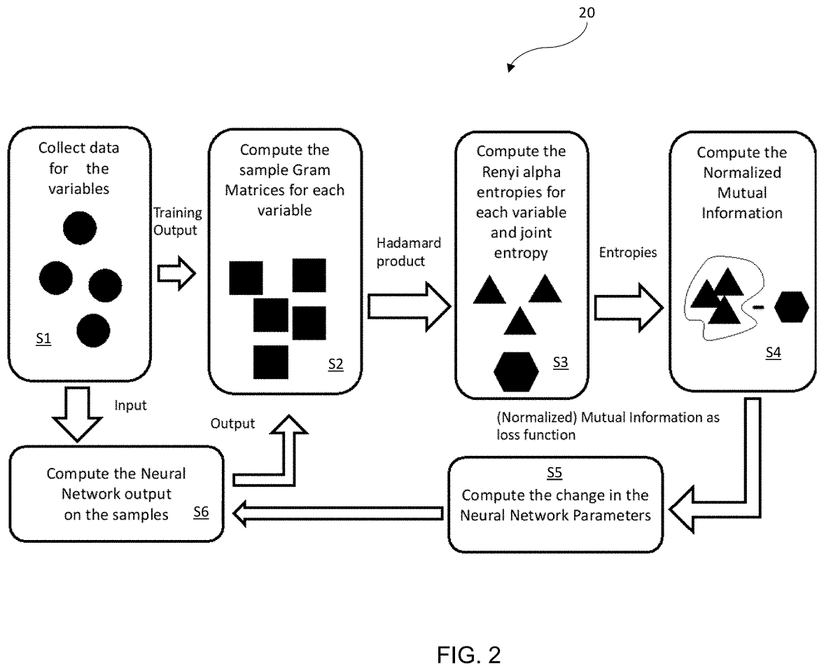

In an embodiment of the present invention illustrated in FIG. 2, the independence measure is a loss function for training a neural network. In the technical problem of training a neural network, the independence measure according to an embodiment of the present invention is the loss function. The loss function can be used, for example, as the measure of the independence between the training data output (or input) and the error. A method 20 of determining the independence measure and using it as a loss function for training a neural network comprises The method comprises the following steps:
S1) Collect the data for the variables;
S2) Compute the Gram matrices for each variable and each sample;
S3) Compute the Renyi alpha entropies based on the Gram matrices for the single variables and the joint distribution;
S4) Compute the normalization factor, and the mutual information, and compute the normalized mutual information as the dependence/independence measure;
S5) Compute the change in the neural network parameters; and
S6) Compute the neural network output on the samples.

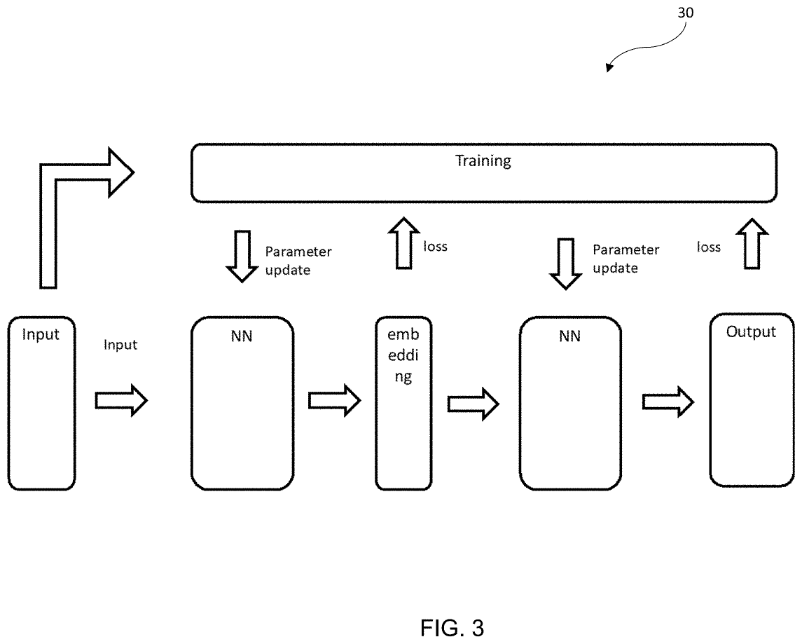

In another embodiment of the present invention illustrated in FIG. 3, the independence measure is implemented in an auto-encoding architecture 30, where the loss function on the embeddings measures the independence of the various variables. This can be used to learn disentangle representations. The training includes parameter updates to the neural network and application of the independence measure as loss functions on the embeddings and output.

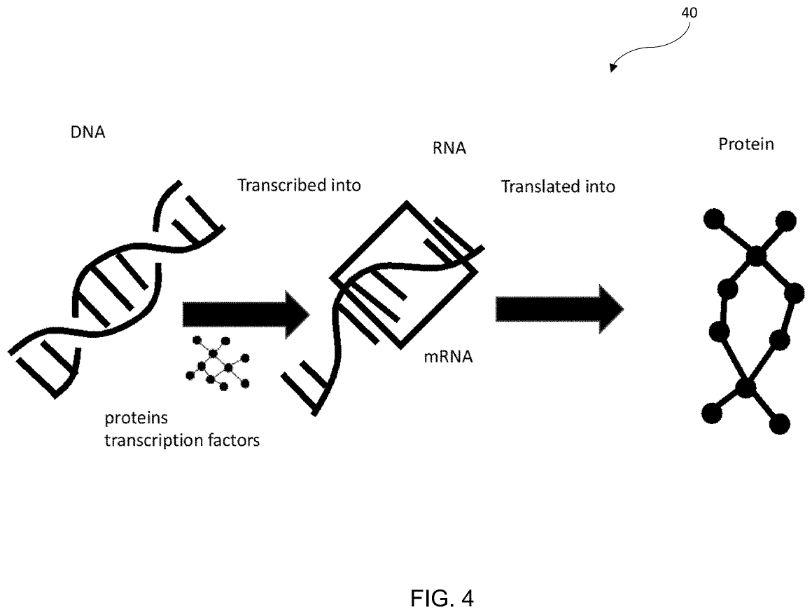

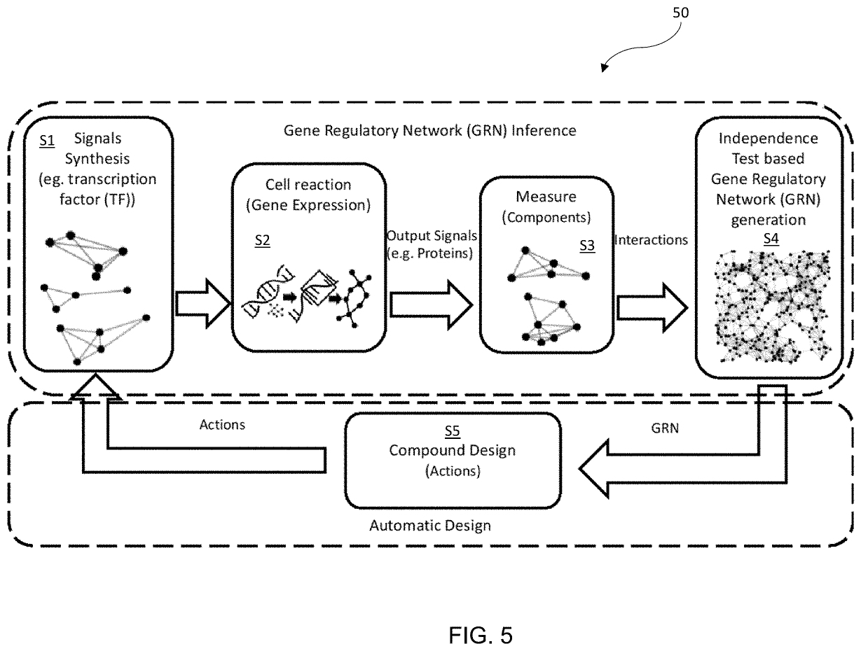

In another embodiment of the present invention, the independence measure is implemented for the technical problem of gene regulatory network inference and automatic experiment design, such as the automatic design of cellular signal design, for example compound design. As shown in FIG. 4, the process of gene expression 40 is a two-step process performed by cells. The process starts from the deoxyribonucleic acid (DNA) sequence and transcribes into an RNA sequence (copy of the DNA gene) by the action of enzyme ribonucleic acid (RNA) polymerase (RNAP) and is modulated by the presence of transcription factors (proteins). The second step takes the result of the DNA transcription and translates it into proteins. The gene expression is used to learn the gene dynamics and the interaction of the cells with the environment, for example, as changes in the environments (e.g., temperature change), presence of external biochemical agents or signals from nearby cells. By measuring the output of these processes, it is possible to understand the cellular and gene interaction. This information and the interactions are collected into the GRN inference (see upper part of FIG. 5).

The regulation of gene expression is a complex process, which involves multiple molecular actors. Further the products of gene expression may affect the gene expression of other genes. The proper understanding of the interactions, possibly involving multiple genes, which elements causes which changes, which gene is activated in a process, is thus a critical and complex computation.

As shown in FIG. 5, an embodiment of the present inventions provides a method 50 to integrate the independence measure to improve the quality of the interaction network generation. In the method 50, the measurements are collected from the cell under study and changes in the environment are performed. Then, the biochemical elements involved in the process are measured. The evaluation of the independence and dependence of the elements are performed according to an embodiment of the present invention, which leads to the construction of the GRN. Based on this information, a new experiment is initiated based on the area where there is no information, by removing or adding elements based on the GRN. The recoded measures and signals are also used to identify GRN structure over different cells. The method 50 includes the following steps:
S1) Signals synthesis (e.g., transcription factor (TF));
S2) Determine cell reaction (e.g., gene expression) outputting signals (e.g., molecules, proteins or other compounds);
S3) Measure a product of the interaction between elements in a biological or cellular process, for example, by measuring pairwise interaction between output signals (e.g., from each gene or cell to determine pairwise dependence or independence between any two genes or cells);
S4) Generate the GRN using the independence measure according to embodiments of the present invention; and
S5) Compound design (Actions). One example of an action is to change the experiment or to change the compound to be tested in the next iteration. Another example of action is to change the temperature of the experiment or the use of additional chemical or biological elements in the experiment.

The technologies used for the input and measurement could be based on next generation sequencing (NGS) technologies (e.g., RNA-seq) or on chromatin immuno-precipitation followed by sequencing (ChIP-seq). The GRN is a graph defined by the vertices V and the edges E, G=(V,E), with E=V×V. Nodes represent the expression level of the genes, while edges represent the presence of interaction between genes. The GRN is built from a set of measurements D of size N×G, where N is the expression measurements on different conditions and G is the number of genes. A typical measure is the Pearson correlation between two measurements (see Huynh-Thu, et al., “Gene Regulatory Network Inference: An Introductory Survey,” ArXiv:1801.04087 [q-Bio], December (2018), which is hereby incorporated by reference herein):

$$corr ( {v}_{i} , {v}_{j} ) = {ρ}_{ij} = \frac{{v}_{i}^{T} {v}_{j}}{‖ {v}_{i} ‖ ‖ {v}_{j} ‖}$$

The network is then built using a threshold on the correlation. This approach has the disadvantage of not revealing if the two genes are correlated for the effect of a third gene. For example, gene g1 and gene g2 may interact because of the effect of gene g3, so they may be correlated, but it is not possible to distinguish this with respect to a direct interaction of genes g1−g2. Also, direct and indirect interactions are not distinguished. If gene g3 interacts with gene g2 and gene g2 interacts with gene g1, then gene g1 and gene g3 are correlated, but the correct structure is not revealed.

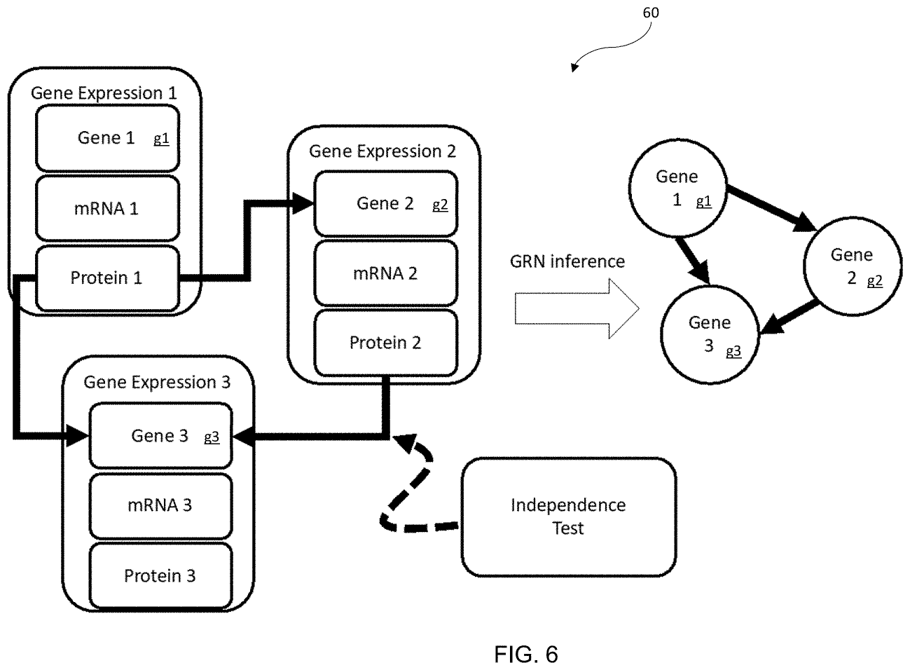

These problems can be overcome by using the independence measure according to an embodiment of the present invention as shown in the method 60 of FIG. 6, by logically modelling the gene expression with a probability distribution and then evaluating. For example, it could be very likely that gene g1 does not interact with gene g3 individually, and gene g2 does not interact with gene g3 individually, but that gene g3 is influenced by the combination of genes (g1, g2). In this case, the measure according to an embodiment of the present invention it is possible to specify that I(g3; (g1,g2))>0, but I(g3; g1)=0 and I(g3; g2)=0.

The method allows to measure the independence and thus distinguish the direct and indirect interaction of the genes. For example, if g2->g3->g1, then gene g2 only influences gene g1 by gene g3, and this is not a direct interaction. Using the measure according to an embodiment of the present invention, it is possible to simply test that the conditional dependence of gene g1 and gene g2 given gene g3 (i.e., I(g1; g2|g3)) is zero, which means that the measure can advantageously distinguish direct interaction from indirect interaction.

An alternative approach is to use information theoretic scores, which measure the mutual information between two quantities (X,Y), which in this case are the genes: MI[X,Y]=H(X,Y)−H(X)−H(Y). In these approaches, the probability distributions of X and Y are evaluated using the empirical distributions from the samples. Using the independence measure according to an embodiment of the present invention improves on these approaches by not requiring to estimate the probability distribution of the two quantities X,Y.

Embodiments of the present invention provide to construct a new GRN, called a High Order GRN, where each node is a combination of genes and its dependence is measured by designing multi-gene experiments. These networks can be derived from statistical evaluation of the dependence of multiple variables in accordance with the method according to an embodiment of the present invention, without been affected by the curse of dimensionality.

Embodiments of the present invention enable the following technical advantages and improvements:
1) More accurate and reliable GRN construction based on the independence measure.
2) Scalability to a large number of variables along with more consistent results for a wide range and number of variables (e.g., genes).
3) Less need for physical experimentation through a more accurate network.
4) Ability to use a large number of targeted design experiments that do not require human intervention.
4) Other technical advantages and improvements in the field of application (e.g., higher probability of designing better vaccines, drugs or protein/chemical compounds with desired characteristics (e.g., a low poison level).

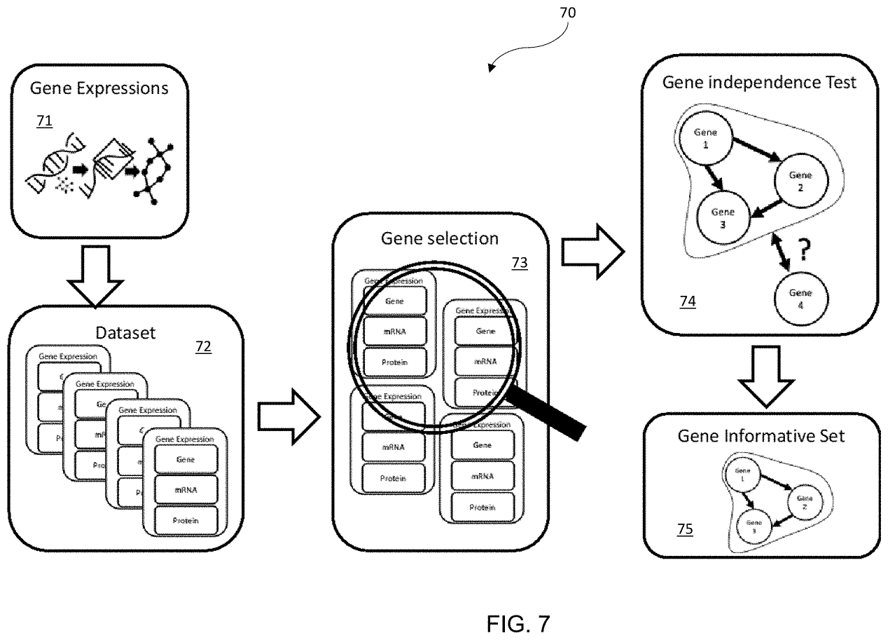

In an embodiment of the present invention illustrated in FIG. 7, a method 70 for informative gene selection uses the independence measure as a gene independence test 74. In this embodiment, the measure of gene expression levels 71 is used for classifying biological samples or design treatment strategies for certain diseases. The gene expression levels 71 can be input into a dataset 72, from which a gene selection 73 can be made. One way to measure gene expression is using a DNA microarray, which allows to measure a large number of gene expressions simultaneously. Biologically, the goal is to identify the set of genes which are active in the presence of a specific disease. This set of genes represents the most informative set 75.

One approach which can be used for an embodiment of the present invention is to measure the prediction performance of a subset of genes as a Lasso problem. Another alternative approach uses adaptive elastic net with conditional mutual information (see Yang, Xin-Guang, et al., “Informative Gene Selection for Microarray Classification via Adaptive Elastic Net with Conditional Mutual Information,” ArXiv:1806.01466 [Cs, Math, Stat], June (2018), which is hereby incorporated by reference herein) as follows: I(X,Y|Z). This method and other approaches based on information theory requires to estimate the probability distribution from the sample data.

In the embodiment of the present invention illustrated in FIG. 7, the gene independence test 74 uses the independence measure to select the genes that have a dependency, while excluding the genes that are independent of the expression process. In the gene independence test 74, it is measured whether an additional gene Xk+1 is independent of the previous selected genes: p(X1, . . . Xk,Xk+1)=p(X1, . . . Xk)p(Xk+1). The order of selection is decided based on the individual gene expression level.

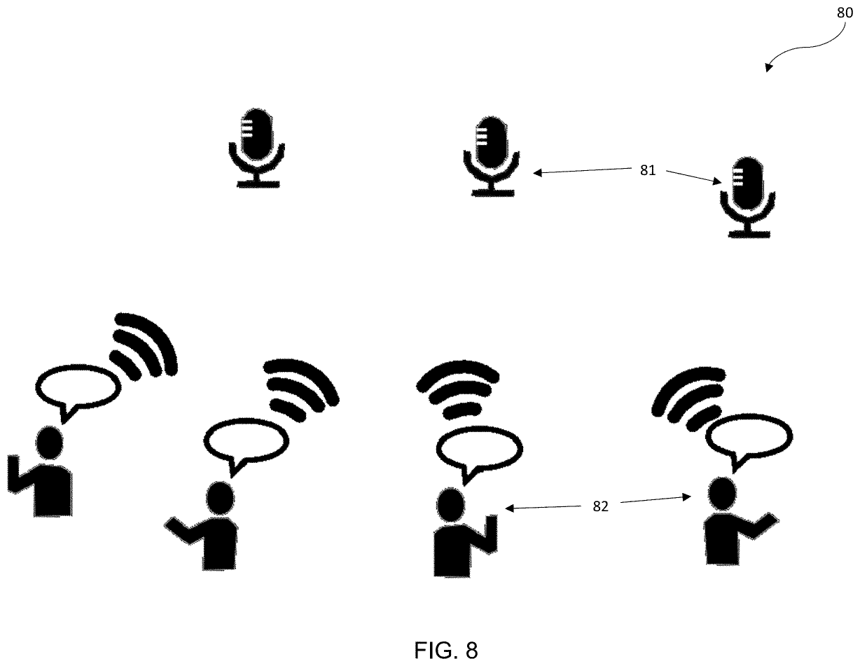

In an embodiment of the present invention illustrated in FIG. 8, the independence measure is applied in a system 80 for blind source separation in signal processing for source separation in noisy and possibly distorted environments. The technical problem of blind source separation occurs, for example, when there are multiple noise sources such as speakers 82 in a room with microphones 81 that are placed to separate the individual speakers 82.

In an embodiment of the present invention illustrated in FIG. 9, the independence measure is applied in a system 90 for blind source separation in petroleum exploration using wave propagation (e.g., a seismic survey). In geographical exploration, a wave (original signal Z) from a signal source 92 propagates through a medium comprising layers a, b, c, d of different material, for example, sand, rock, water, gas/air, petroleum, etc. Thus, in geographical exploration the variables can be geological composition and distance represented by a mixed (or corrupted) signal Z. Receivers denoted by the microphones 94 receive the mixed signal X which is analyzed by an analyzing system denoted by the truck 95, which performs, e.g., independent component analysis (ICA) with the measure according to an embodiment of the present invention, to reconstruct the original signal Z from the mixed signal X. In this embodiment, the goal is to reconstruct the individual source from the measurements, without having the control on the source signal. Possible approaches also include to use principal component analysis (PCA) using singular value decomposition (SVD) or ICA. If Z is the original source signal, the measure to the receiver is considered to be X=AZ+E, where A represent the distortion (or mixing matrix) and E the noise. The reconstruction is done using a linear transformation Y=WX. Similarly, it is possible to use a non-linear architecture, where the Y=ƒ_{W}(X). The original signals can be reconstructed using the mutual information (or total correlation) as follows:

$${I}_{W} ( {y}_{1} , … , {y}_{M} ) = {\sum }_{i = 1 , … , M} H ( {y}_{i} ) - H ( y ) ∼ {\sum }_{i = 1 , … , M} ( H ( {y}_{i} ) - H ( {x}_{i} ) ) - log ‖ W ‖$$

where the ICA solves:

$${min}_{W} {I}_{W} ( {y}_{1} , … , {y}_{M} )$$

where the entropies are computed from the empirical distributions.
This approach suffers from the error in reconstruction of the empirical distribution and the curse of dimensionality.

This approach suffers from the error in reconstruction of the empirical distribution and the curse of dimensionality which can be overcome by applying the independence measure according to an embodiment of the present invention, in particular in the ICA to evaluate the independence of the reconstructed signal. The technical advantages and improvements in the blind source separation embodiments include:
1) More accurate separation of sources, which allows to more clearly listen to multiple speakers or measure the propagation delay of seismic or other waves for petroleum exploration.
2) Scalability to a large number of sources and high dimensionality of the signal, for example providing to use multi modal waves (e.g., different frequencies and/or different oscillations/polarizations).
3) Better distinction of the sources and thus a better description of the location of various material for petroleum exploration or better differentiation of the speaker for speaker separation.
4) Less physical exploration required, and potential to use a large number of sources and measurements.
5) Less noisy separation of speakers or sources.

Describe the Inventive Steps and point out what the actual technical improvements are compared to existing technologies.

Applicable to all applications which utilize a measure of the dependence and independence of variables, embodiments of the present invention enable the following technical advantages and improvements:
1) Improved accuracy and reliability.
2) Scalability to a large number and/or higher-order of variables.
3) A method that computes independence (or degree of dependence) of variables, using information theoretical mutual information (total correlation) for more than two (multiple) variables and/or group of variables from the data kernel Gram matrices. The method can include a number of unconventional features such as:
   a. A measure that uses the Hadamard products of the data kernel Gram matrices, but not as kernels for prediction.
   b. A measure of the content of information on the variable using (Renyi alpha) entropy.
   c. A comparison of the sum of entropy of all single variables (from the kernel matrix of the single variable) against the joint entropy of all variables (Hadamard product of the kernel Gram matrices). The Renyi alpha entropy function is used to evaluate entropy from each of the variables (because a Gram matrix is computed for each variable). The Hadamard product is also used to evaluate the joint entropy.
   d. Combination of the features including the computation of Gram matrices from samples, their element-wise product (e.g., Hadamard product), and the computation of the entropy (the measure of the information contained in the matrix), potentially along with normalization to obtain interpretability.
4) Providing a measure that is strictly between [0,1] for improved interpretability. For example, 0 can indicate complete independence, whereas 1 can indicate straightforward functional dependence. The normalization factor can be computed based on an upper bound on the mutual information measure (using the Shearer's inequality).
5) Enabling to remove indirect interactions and to quantify high-order interactions.
6) Providing an independence measure that is automatically differentiable. Since the measure is differentiable using automatic differentiable packages, it can be used in recent machine learning frameworks, and can be integrated as a loss function in various machine learning tasks.
7) Utilizing the automatically differentiable independence measure as a loss function for training neural networks, which allows, for example, to be robust to covariant shift of the input distribution. By usage of the automatically differentiable property of the independence measure to train neural networks, the new loss function is robust against both the covariate shift and the label noises. In contrast, existing solutions address this problem either from an optimization perspective or perform domain adaptation, which requires unlabeled data from target domain, while embodiments of the present invention provide that the loss is fully unsupervised and is totally independent of target data.
8) Less affected by the curse of dimensionality by using the Gram matrices as building blocks and the eigenvalues of the Hadamard product of the Gram matrices. The procedure is more stable numerically, and also less affected to the curse of dimensionality. In other words, the accuracy of the measure is more stable with larger dimensions (e.g., amount of variables to compare).

Other solutions such as those described above and below which do not use the independence measure, for example, solutions including Kullback-Leibler (KL) divergence, do not provide for the foregoing technical advantages or improvements.

In an embodiment, the present invention provides a method for determining an independence measure of variables, the method comprising the following steps:
1) Collection of the data to form the variables;
2) Build the sample kernel Gram matrices for the variables; and
3) Compute the Hadamard products of the Gram matrices and the total correlation (mutual information) in its normalized form to obtain the independence measure as a result.

Thus, information from each variable is first computed, and then the entropy from all individual variables is summed. The joint information is then computed also with the Renyi alpha entropy function based on the Hadamard product. Finally, the sum of individual information minus the joint information is determined to be the mutual information.

The method can also include the following steps:
1) Build the network of interactions (e.g., GRN);
2) Provide design actions/signals based on the built network; and
3) Update the neural network parameters based on the computed loss function for independence quantification.

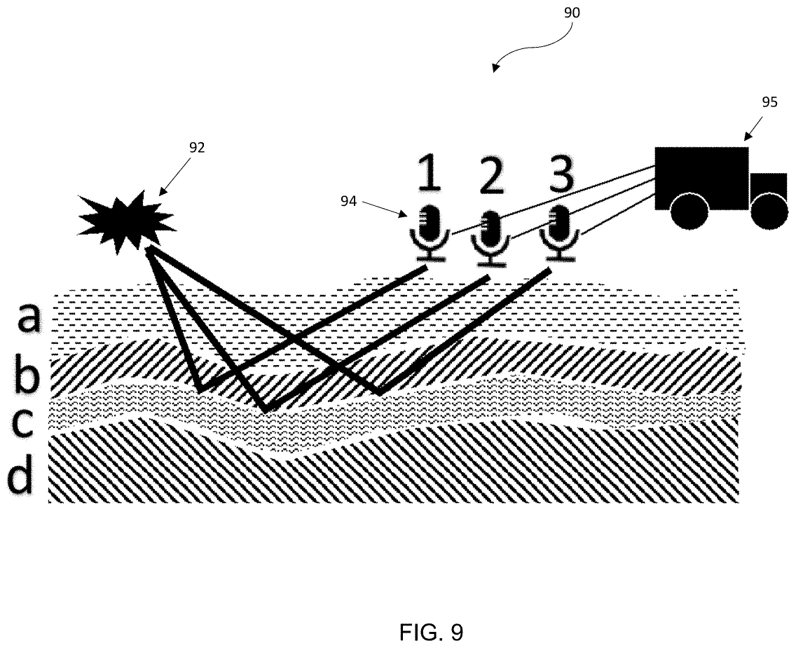

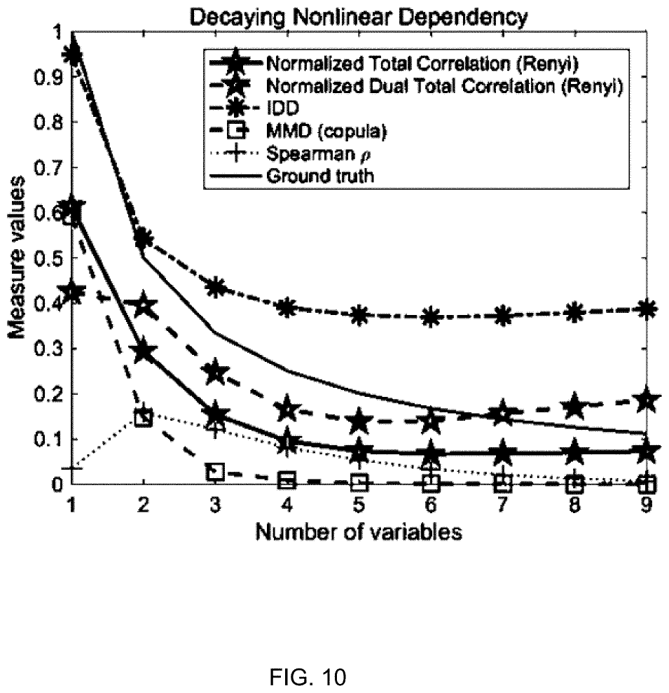

FIG. 10 shows the results of experiments using different independence measures. As shown therein, embodiments of the present invention achieve increased accuracy as they are closer to the ground truth for different numbers of variables and will converge to the ground truth with an increasing number of variables as discussed further below. The normalized total correlation (Renyi) and the normalized dual total correlation (Renyi), which respectively refer to the T* and D* measures, are according to embodiments of the present invention and are compared against different methods IDD, MMD and Spearman that compute correlation or dependence, as well as against the ground truth.

Embodiments of the present invention can be applied for understanding of biological systems, such as in the construction of the GRN and gene selection, for example, for disease treatment in medical applications, or for signal separation in bioinformatic applications or other applications such as speaker identification, source separation and geological exploration. An embodiment of the present invention can also be applied in artificial intelligence for drug development (AIDD) to infer high-order relationships, for example, to infer drug side effects or drug discovery.

In the following, embodiments of the present invention are described in further detail, along with a quantitative showing from experiments on datasets which demonstrates the technical advantages and improvements provided thereby compared to other and existing solutions. To the extent different terminology is used below and would be understood by an ordinarily skilled artisan to correspond to respective features discussed above, it is to be understood that such descriptions of these same features are applicable to the embodiments described above as well, despite the different terminology.

Embodiments of the present invention provide an independence measure based on the eigenspectrum of a Hermitian matrix of the projected data in a reproducing kernel Hilbert space (RKHS). The independence measure is automatically differentiable and provides a robust cost function to train neural networks by encouraging the independence between input x and prediction error e. Advantageously, it has been found that there is equivalence between this loss function and the well-known minimum error entropy (MEE) criterion. Experimental results demonstrate that the independence measure is more powerful than well-known independence statistics like kernel canonical correlation analysis (KCCA) and Hilbert-Schmidt independence criterion (HSIC). Moreover, the loss function based on the independence measure is robust against both covariate shift (in transfer learning) and non-Gaussian noises.

Embodiments of the present invention recognize that the technical problem of measuring (or testing) the dependence (or independence) of random vectors and the problem of robust machine learning are not isolated. Specifically, the independence measure according to embodiments of the present invention is represented as follows: I_{α}*(x,y), in particular a matrix-based Renyi's α-order normalized mutual information, on random vectors x∈ [symbol] ^{p} and y∈ [symbol] ^{q}. The independence measure I_{α}*(x,y) is automatically differentiable and can be used explicitly as a cost function to train neural networks by encouraging the independence between input x and prediction error e (i.e., min I_{α}*(x,e)). The robustness of this loss function has been demonstrated against covariant shift and non-Gaussian noises.

Embodiments of the present invention enable the following contributions as technical advantages and improvements over existing solutions:
1) Measuring and testing the dependence (or independence) with the matrix-based Renyi's α-order entropy function. The new estimator avoids the estimation of the underlying probability density function (PDF) of data, and is operated on the eigenspectrum of a (normalized) Hermitian matrix of the projected data in an RKHS. This intriguing property makes the novel measure can be easily applied to real-world high-dimensional data with complex data distributions.
2) By encouraging the independence between input x and the prediction error e (i.e., minimizing of the independence measure I_{α}*(x; e)), the independence measure provides an improved loss function which provide more robust machine learning against the shift of the input distribution (the covariate shift). Since the loss function is automatically differentiable it can be simply embedded into a neural network.
3) By establishing the equivalence between the criterion of minimum mutual information (min I(x; e)) and the criterion of minimum error entropy (MEE) (min H(e)), it is provided that the loss function is robust to non-Gaussian noises as well, especially those with long tails. This has been empirically demonstrated as discussed below. Theoretical analysis (with respect to the well-known mean square error (MSE) criterion) is also provided below.
4) As indicated by a power test, the independence measure according to embodiments of the present invention is more powerful than other notable dependence/independence measures, such as the HSIC, KCCA and the distance of covariance (dCov), in identifying the independence relationships and discovering highly non-linear relationships. Experimental results in real-world datasets also demonstrate the improved performance gained by using the independence measure as a cost function in case of covariate shift and non-Gaussian noises.

The technical problem addressed by embodiments of the present invention can be formulated as follows: given two random vectors x∈ [symbol] ^{p} and y∈ [symbol] ^{q} of arbitrary dimensions p and q, respectively, and given n independent realizations of the random vectors represented by matrices X∈ [symbol] ^{n×p} and Y∈ [symbol] ^{n×q}, the goal is to have a measure to identify the complex (and possibly non-linear) statistical associations between x and y. Preferably, the measure is provided strictly between [0, 1] for improved interpretability. Meanwhile, it is also expected that the statistic is powerful to support a test of independence with the following null and alternative hypotheses:

H₀:Pr(x,y)=Pr(x)Pr(y)

H₁:Pr(x,y)≠Pr(x)Pr(y),

where Pr refers to the probability distribution.

As related work, the following is a discussion of unsupervised covariate shift, random vector dependence.

The technical problem of unsupervised covariate shift is formulated as follows: let (x; y) be a pair of random variables such that x∈ [symbol] ^{p} and y∈ [symbol] , with a joint distribution P_{source}(x; y), such that x denotes the instances and y denotes the desired responses, with the goal, given a training set drawn from P_{source}(x; y), to learn a model predicting y from x that works well on a different, α-priori unknown target distribution P_{target}(x; y). In the scenario of covariate shift, the assumption is that the conditional label distribution is invariant (i.e., P_{target} (y|x)=P_{source}(y|x)) but the marginal distributions of input P(x) are different between source and target domains (i.e., P_{target}(x)≠P_{source}(x)). An embodiment of the present invention advantageously provides improvements for unsupervised covariate shift, where there is no access to samples x or y from the target domain.

With respect to random vector dependence, various measures have been proposed to quantify the dependence between two random vectors. Common measures of linear dependence, such as the RV coefficient and canonical correlation analysis (CCA), are computationally efficient and theoretically well-understood, but consider only a limited class of association patterns, like monotonically increasing functions. The development of non-linear dependence measures is technically challenging because of the radically larger amount of possible association patterns. Notable non-linear statistical dependence measures include the dCov, HSIC, and the randomized dependence coefficient (RDC). Particular interest can be paid to RDC, which measures the dependence of X and Y as the largest canonical correlation between k randomly chosen non-linear projections of their copula transformations. Surprisingly, RDC can be implemented with 5 lines of R or MATLAB code.

Giraldo, Luis Gonzalo Sanchez, et al., “Measures of entropy from data using infinitely divisible kernels,” IEEE Transactions on Information Theory 61, no. 1, pp. 535-54 (2014), which is hereby incorporated by reference herein, describes a matrix-based Renyi's α-order entropy function and demonstrates the way to measure the mutual information between two random variables using the eigenspectrum of (normalized) Gram matrices. In contrast to prior work, an embodiment of the present invention normalizes the mutual information such that the independence measure is strictly between [0, 1] for improved interpretability. A simple test was used to determine that the empirical estimates of the normalized mutual information indicate a statistically significant improvement. Simulation results suggest that the measure according to embodiments of the present invention is more powerful than existing approaches, such as HSIC and KCCA. It is demonstrated below how the independence measure is automatically differentiable and can be used as a robust cost function for both regression and classification in case of covariate shift and non-Gaussian noises. For example, by replacing HSIC with the independence measure according to an embodiment of the present invention, an obvious performance gain is obtained on both synthetic and real-word datasets. A novel insight on the criterion of min I(x; e) is also discussed below. By equating min I(x; e) with min H(e), it is demonstrated that the novel learning criterion using the independence measure according to embodiments of the present invention is also robust to non-Gaussian noises.

Embodiments of the present invention use the independence measure I(x; y), which can also be referred to as the mutual information or independence test, to quantify the dependence between two random vectors x and y. Further, in order to make sure the measure is strictly between [0, 1], I(x; y) is normalized by max{H(x); H(y)} as follows:

$${I}^{*} ( x ; y ) = \frac{I ( x ; y )}{max \{ H ( x ) ; H ( y ) \}} . \qquad ( 1 )$$

There are also other candidate normalization terms, such as ½(H(x)+H(y)) and \sqrt{H(x)H(y)}. For example, the former gives equal weight to H(x) and H(y), whereas the latter I(x; y)/\sqrt{H(x)H(y)} can be interpreted somewhat as an analogy to the correlation coefficient ρ=cov(x; y)/\sqrt{var(x)var(y)}. As an example for the following discussion, max{H(x); H(y)} is chosen because 1−max{H(x); H(y)} is a distance metric such that I(x; y)/max{H(x); H(y)} is a similarity metric.

In accordance with an embodiment of the present invention, it is advantageous as discussed below to estimate dependence with the matrix-based Renyi's α-order entropy function. To estimate I*(x; y) from samples {(xᵢ,yᵢ)}ᵢ₌₁ⁿ, one needs to estimate the mutual information term I(x; y) and the entropy term H(x) or H(y). According to Shannon's information theory, I(x; y) is defined over the joint probability distribution of x and y (i.e., p(x; y)) and their respectively marginal distributions (i.e., p(x) and p(y)). Specifically, as follows:

$$I ( x ; y ) = \int \int p ( x , y ) log ( \frac{p ( x , y )}{p ( x ) p ( y )} ) d xdy = - \int ( \int p ( x , y ) dy ) log p ( x ) d x - \int ( \int p ( x , y ) dx ) log p ( y ) d y + \int \int p ( x , y ) log p ( x , y ) dxdy = - \int p ( x ) log p ( x ) d x - \int p ( y ) log p ( y ) d y + \int \int p ( x , y ) log p ( x , y ) dxdy = H ( x ) + H ( y ) - H ( x , y ) , \qquad ( 2 )$$

where H(⋅) denote the entropy and H(⋅,⋅) denotes the joint entropy.

According to Eq. (2), on the one hand, one is able to estimate those information-theoretic quantities with Shannon discrete entropy. However, selecting a proper data discretization is a tricky technical problem and an improper discretization strategy may lead to serious estimation error. On the other hand, if the Shannon differential entropy is used, an issue arises naturally on the precise estimation of the (joint) probability distribution functions (PDF). If the prior information on PDF is not available (which is common in data-driven science), the PDF estimation is notoriously difficult in high-dimensional space.

To overcome these technical problems, embodiments of the present invention utilize the matrix-based Renyi's α-entropy function, which suggests similar quantities in terms of the normalized eigenspectrum of the Hermitian matrix of the projected data in the RKHS, thus estimating the entropy and joint entropy directly from data without PDF estimation. In particular, the definitions on entropy and joint entropy in embodiments of the present invention are provided below.

Definition 1: Let κ: χ×χ [symbol] [symbol] be a real valued positive definite kernel that is also infinitely divisible. Given {xᵢ}ᵢ₌₁ⁿ∈χ, each xᵢ can be a real-valued scalar or vector, and the Gram matrix K obtained from evaluating a positive definite kernel K on all pairs of exemplars, that is K=κ(xᵢ,xⱼ), a matrix-based analogue to Renyi's α-entropy for a normalized positive definite matrix A of size n×n, such that tr(A)=1, can be given by the following function:

$${H}_{α} ( A ) = \frac{1}{1 - α} log ( {A}^{α} ) = \frac{1}{1 - α} {log}_{2} ( {\sum }_{i = 1}^{n} {{λ}_{i} ( A )}^{α} ) , \qquad ( 3 )$$

where A is the normalized version of K, i.e., A=K/tr(K), and λᵢ(A) denotes the i-th eigenvalue of A.

Definition 2: Given n pairs of samples (xᵢ,yᵢ)ᵢ₌₁ⁿ, each sample contains two different types of measurements x∈χ and y∈γ obtained from the same realization, and the positive definite kernels κ₁: χ×χ [symbol] [symbol] and κ₂: γ×γ [symbol] [symbol] , a matrix-based analogue to Renyi's α-order joint-entropy can be defined as:

$${H}_{α} ( A , B ) = {H}_{α} ( \frac{A ∘ B}{t r ( A ∘ B )} ) , \qquad ( 4 )$$

where Aᵢⱼ=κ₁(xᵢ,xⱼ)), Bᵢⱼ=κ₂(yᵢ,yⱼ) and A∘B denotes the Hadamard product between the matrices A and B.

Given Eqs. (3) and (4), the matrix-based Renyi's α-order mutual information I_{α}(A; B) in analogy of Shannon's mutual information is given by:

I_{α}(A;B)=H_{α}(A)+H_{α}(B)−H_{α}(A,B). (5)

According to an embodiment of the present invention, the Gram matrices are obtained by the radial basis function (RBF) kernel

$$κ ( {x}_{i} , {x}_{j} ) = exp ( - \frac{{‖ {x}_{i} - {x}_{j} ‖}^{2}}{2 {σ}^{2}} ) .$$

Eqs. (5) and (3) are used to estimate the numerator and denominator of Eq. (1), respectively. The estimates according to embodiments of the present invention advantageously avoid real-valued PDF estimation and have no additional requirement on data characteristics (e.g., continuous, discrete, or mixed), which provides for great potential in a number of applications. The derived quantity of the matrix-based Renyi's α-order normalized mutual information is termed and denoted by I_{α}*.

To demonstrate the power of the independence measure according to embodiments of the present invention for identifying independent relationships and in discovering highly non-linear relationships, two simulations were performed. The independence measure according to embodiments of the present invention (ID was compared with four notable existing dependence measures that have been widely used in machine learning and signal processing communities. These include the KCCA, HSIC, dCov and the Cauchy-Schwarz quadratic mutual information (QMI_CS).

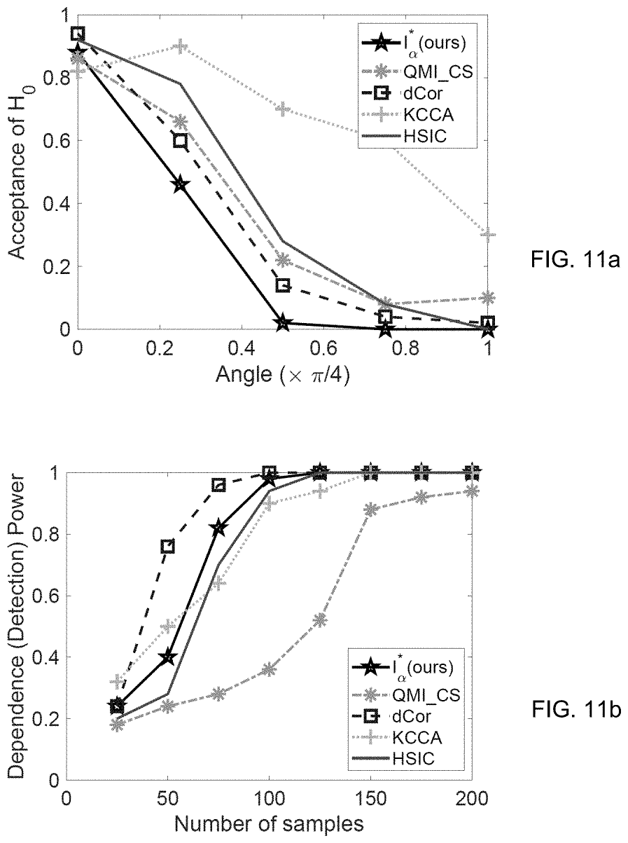

The first test dataset was generated. First, N i.i.d. samples were generated from two randomly picked densities corresponding to the independent component analysis (ICA) benchmark densities. In the example shown in FIG. 11a, the first density is a Student's t distribution (a standard distribution in statistics with a closed-form expression where the shape of this distribution is controlled by its degree of freedom) with 3 degree of freedom (DOF), whereas the second density is also a Student's t distribution but with 5 DOF. Second, these random variables were mixed using a rotation matrix parameterized by an angle θ, varying from 0 to π/4. Third, it was added d−1 extra dimensional Gaussian noise of zero mean and unit standard deviation to each of the mixtures. Finally, each resulting vector was multiplied by an independent random d-dimensional orthogonal matrix. This causes the resulting random vectors to be dependent across all observed dimensions.

The second test dataset was generated by a matrix X∈ [symbol] ^{N×5} having been generated from a multivariate Gaussian distribution with an identity covariance matrix. Then, another matrix Y∈ [symbol] ^{N×5} was generated Yₘₗ=log(Xₘₗ²), m=1, 2, . . . , N, l=1, 2, . . . , 5.

In each test dataset, each tested measure (I_{α}*, HSIC, KCCA, dCov, QMI_CS) was compared with a threshold computed by sampling a surrogate of the null hypothesis H₀ based on shuffling samples in Y with k times (k=100). The correspondences between xᵢ and yᵢ are broken by the random permutations. The threshold is the estimated quantile 1−τ where τ is the significance level of the test (Type I error). If the estimated measure is larger than the computed threshold, the null hypothesis was rejected and the existence of an association between X and Y was argued, and vice versa.

The above procedure was repeated for five hundred independent trials. FIGS. 11a and 11b demonstrate the average acceptance rate of the null hypothesis H₀ (in test data I with respect to different rotation angles θ) and the average detection rate of the alternative hypothesis H₁ (in test data II with respect to different number of samples). Intuitively, in the first test dataset, a zero angle means the data are independent, while dependence becomes easier to detect as the angle increases to π/4. Therefore, a desirable measure is expected to have acceptance rate of H₀ nearly to 1 at θ=0. The rate is expected to rapidly decay as 0 increases. In the second test dataset, a desirable measure is expected to always have a large detection rate of H₁ regardless of the number of samples.

Embodiments of the present invention further provide for robust learning under covariate shift using an independence criterion for unsupervised covariate shift. In particular, embodiments of the present invention provide the independence measure as a loss function which measures the independence between the input sample space and its corresponding error space. Advantageously, the residual between the output of model and the desired result is independent of the input. In particular, an embodiment of the present invention uses the following learning problem:

$${min}_{θ} I ( X ; Y - {f}_{θ} ( X ) ) \qquad ( 6 )$$

This independence measurement object function is in contrast to typical loss functions, which can decompose as a sum of losses over each individual sample. One weakness of this loss function is that I(X, Y−ƒ_{θ}(X)) is exactly the same for any two functions ƒ_{θ}_{₁}(X), θ_{θ}_{₂}(X) that differ only by a contestant, because when adding a constant, it is not possible to change the angle of the space. In order to solve this problem, an embodiment of the present invention provides to calculate the empirical mean of error for the training set and add this bias in the output of the model. As long as the Gram matrix A_{σ}_{ₓ}(x), A_{σ}_{ₑ}(e) and the eigenvalue of Gram matrix are differentiable, it's straightforward to take the gradient of the I_{α}(x; e) with any deep learning framework, which can help to take the gradient automatically. Preferably, Pytorch is used as the deep learning framework, because it is capable of providing gradients of eigenvalues with respect to the underlying matrix. A general gradient-based method for I_{α}(x; e) which can be used in an embodiment of the present invention is illustrated in Algorithm 1 which is set forth below.

Algorithm 1: Learning I_{α}(x:e) loss

| | |
|---|---|
| 1: | Input: samples (xᵢ, yᵢ)ᵢ₌₁ⁿ, kernel size σₓ, σₑ, α for Renyi’s |
| | α-entropy, neural network ƒ_{θ} parameterized by θ. |
| 2: | Repeat: |
| 3: | Sample mini-batch (xᵢ, yᵢ)ᵢ₌₁^{m} |
| 4: | Compute errors eᵢ^{θ} = yᵢ − ƒ_{θ}(xᵢ) |
| 5: | Compute the Gram matrix with Gaussian kernel for (xᵢ)ᵢ₌₁^{m} and |
| | its corresponding errors (eᵢ)ᵢ₌₁^{m} and normalize them A_{σ}_{ₓ}(x), A_{σ}_{ₑ}(e) |
| 6: | Compute Renyi’s α-entropy for input samples H_{α}(x) and H_{α}(e) |
| | according to Eq. (3). |
| 7: | Compute I_{α} (x, e) loss according to Eq. (5) and normalize it |
| | with Eq. (1). |
| 8: | Update θ ← Optimize(I(x; e)) |
| 9: | Until convergence. |
| 10: | Compute the estimated source bias: |
| 11: | $b ← \frac{1}{N} {Σ}_{i = 1}^{n} {y}_{i} - \frac{1}{N} {Σ}_{i = 1}^{n} {f}_{θ} ( {x}_{i} ) \qquad$ |
| 12: | Outputs ƒ(x) = ƒ_{θ}(x) + b. |

Experiments were performed to demonstrate and validate the robustness against covariate shift when using the independence measure according to embodiments of the present invention as the loss function. The experiments used the MNIST and fashion MNIST datasets.

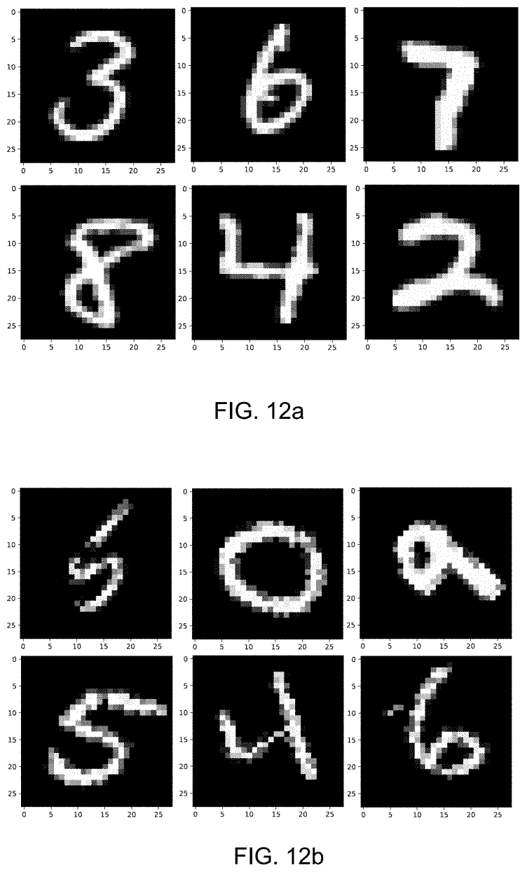

In the experiment using the MNIST dataset, the goal was to test the performances of different cost functions on the MNIST dataset with covariate shift. The MNIST dataset was trained as the source domain, and the target domain consists of digits which are rotated by an angle θ sampled from a uniform distribution over [−π/4,π/4]. FIGS. 12a and 12b respectively show the source and target images for MNIST dataset.

Training was compared with cross entropy (CE) loss, HSIC loss, H_{α}(e) loss and I_{α}(x; e) loss. H_{α}(e) loss and I_{α}(x; e) loss are each losses according to embodiments of the present invention. I_{α}(x; e) uses the independence measure on x and e (two variables) while H_{α}(e) uses the independence measure on e (one variable). The architecture of the model in accordance with an embodiment of the present invention is as follows: there are three layers, including two convolutional layers and one fully connected layer, and the kernel size of the convolutional layers is 5×5. Batch normalization and a max-pooling layer was also added after each convolutional layer. The activation function is rectified linear unit (ReLU). The number of units for the first convolutional layer was 16 and the number of units for the second convolutional layer was 32. The batch size for all the experiments was 128 and optimization was performed using the Adam optimization algorithm.

For I(x; e) loss, parameter a for Renyi's α-entropy was set as 2 and the a for the error space and input space are all 1. For error entropy H(e) loss, the same parameters were used as set for I(x,e) loss. For HSIC loss, a was set for the error space as 1 and for the input space as 22. The results are depicted in Table 1 below. There it can be seen that, for all different loss functions, moving to the target domain induces a large drop in accuracy. It can also be seen that using H_{α}(e) and I_{α}(x,e) loss according to embodiments of the present invention provides better performance not only on the source dataset but also on target dataset compared to using the HSIC loss and cross-entropy loss.

TABLE 1
Test accuracy (%) for MNIST dataset

| | MNIST | |
|---|---|---|
| Method | Source | Target |
| CE | 99.25 ± 0.006 | 87.54 ± 0.076 |
| HSIC | 99.26 ± 0.003 | 87.93 ± 0.134 |
| H_{α}(e) | 99.26 ± 0.001 | 88.11 ± 0.069 |
| I_{α}(x; e) | 99.27 ± 0.004 | 87.95 ± 0.144 |

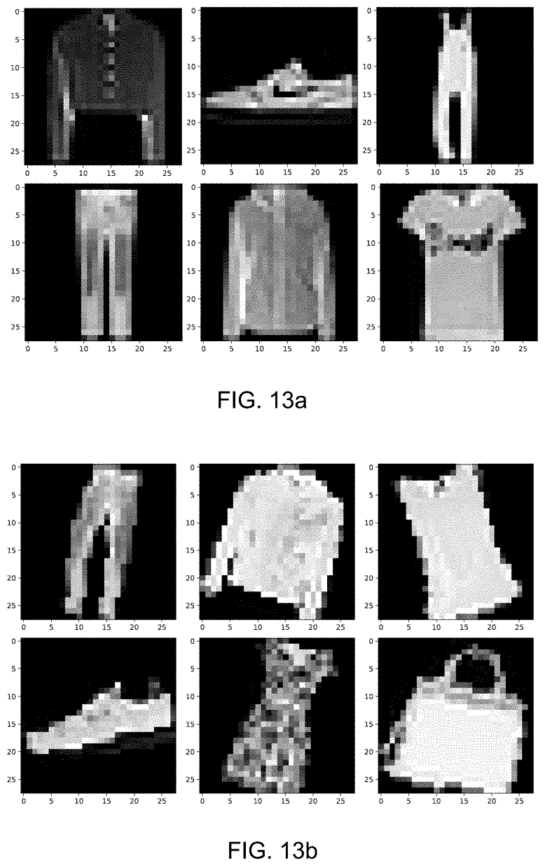

Another experiment testing the performance of the cost function in accordance with embodiments of the present invention was conducted with fashion MNIST dataset, which is more technically challenging to use than the MNIST dataset as the size of each grayscale image is 28×28, associated with a label from 10 classes. The training set has 60,000 images and the test set has 10,000 images. Similar to the rotating of the MNIST dataset, each image was rotated with an angle θ from [−20, 20] and treated as the target dataset. The source and target images for the fashion MNIST is shown in FIGS. 13a and 13b, respectively. The same model as discussed above for the rotating MNIST experiment was used. When training with different loss functions, the batch size was 128. It was found that for I_{α}(x,e) loss, when α=5 and σₓ=σₑ=1, better results were achieved. The parameter α for I_{α}(x,e) and H_{α}is the same. For HSIC loss, σ was set for the error space as 1 and for the input space as 22. Table 2 below shows the results for rotating fashion MNIST, which was repeated ten times for each experiment. It can be seen that H_{α}(e) and I_{α}(x,e) loss in accordance with embodiments of the present invention perform much better than standard cross entropy loss and show the strong probability for robustness to covariate shift in contrast to cross entropy and HSIC loss.

TABLE 2
Test accuracy (%) for Fashion MNIST

| | Fashion MNIST | |
|---|---|---|
| Method | Source | Target |
| CE | 90.90 ± 0.002 | 73.73 ± 0.086 |
| HSIC | 91.03 ± 0.003 | 76.56 ± 0.034 |
| H_{α}(e) | 91.10 ± 0.013 | 75.48 ± 0.069 |
| I_{α}(x; e) | 91.17 ± 0.040 | 76.79 ± 0.040 |

Embodiments of the present invention provide for robust learning under non-Gaussian noise. There is equivalence between min I(x; e) and min H(e). Given input signal x and desired response y, and the prediction model is f, the following relationship applies:

y=ƒ(x)+e, (7)

where e is the prediction error.

The criterion of min I(x; e) is introduced above and its implementation with respect to HSIC and the independence measure according to embodiments of the present invention is discussed. In the following, the equivalence of min I(x; e) and min H(e) is proven, where the latter is exactly the well-known minimum error entropy (MEE) criterion. In fact, the following relationships apply:

$$\begin{matrix}I ( x ; e ) = H ( e ) - H ( e \vert x ) \\ = H ( e ) - H ( e + f ( x ) \vert x ) \\ = H ( e ) - H ( y \vert x ) ,\end{matrix} \qquad ( 8 )$$

in which the second equality is by the property that given two random variables ξ and η, then for any measurable function h, there exists H(ξ|η)=H(ξ+h(η)|η).

Therefore, min I(x; e) is equivalent to min I(e). This is simply by the fact that the conditional entropy of y given x, i.e., H(y|x), is a constant that is purely determined by the training data.

To further demonstrate the advantages of the independence measure according to embodiments of the present invention, an experiment for synthetic data was also performed. The experiment included experimenting with fitting a linear model. The underlying model in the experiments is y=β^{T}x+ϵ where β is a 100 dimension weight vector, which is drawn for each experiment from a zero mean Gaussian distribution with σ=0.1. In the training phase, x is drawn from a uniform distribution over [−1,1]. It was experimented with ϵ drawn from one of three distributions: Gaussian, Laplacian, or a shifted exponential: E=1−e where e is drawn from an exponential distribution exp(1). In any case, E is drawn independently from x. The number of training samples was 1024 and the test set size was 128. For each experiment, different loss function were used as follows: squared-loss, I(x; e) loss, H(e) loss and HSIC loss. The source test set was created in the same manner as the training set, while for target test set, the marginal distribution of input samples was changed from a uniform distribution to a standard Gaussian distribution. The noise was drawn from the same distribution on the source and target datasets for all the cases. A simple linear neural network with one layer was used, with the number of input units being 100, the output unit being 1. It was set a equal to 2, and each experiment was repeated 10 times. It was set σₓ=σₑ=1, and the batch size as 32 with the Adam optimizer.

Table 3 presents the mean square error for different types of noise. With Gaussian noise, I_{α}(x; e) and H_{α}(e) perform similarly to squared-loss regression, and with Laplace noise, I_{α}(x; e) shows better results than HSIC and MSE. For shifted-exponential noise, H_{α}(e) provides a better performance than others.

TABLE 3
Mean square error for synthetic data

| | Gaussian noise | | Laplace noise | | Shifted-exponential noise | |
|---|---|---|---|---|---|---|
| Method | Source | Target | Source | Target | Source | Target |
| MSE | 0.95 ± 0.003 | 1.707 ± 0.008 | 1.440 ± 0.013 | 2.225 ± 0.007 | 0.813 ± 0.004 | 1.659 ± 0.006 |
| HSIC | 0.91 ± 0.004 | 1.703 ± 0.006 | 1.463 ± 0.005 | 2.223 ± 0.021 | 0.779 ± 0.005 | 1.639 ± 0.002 |
| H_{α} (e) | 0.94 ± 0.003 | 1.696 ± 0.007 | 1.450 ± 0.008 | 2.193 ± 0.006 | 0.790 ± 0.009 | 1.621 ± 0.007 |
| I_{α} (x; e) | 0.93 ± 0.002 | 1.704 ± 0.008 | 1.420 ± 0.030 | 2.217 ± 0.013 | 0.781 ± 0.004 | 1.657 ± 0.009 |

In the following, deeper insights into robustness of min H(e) against non-Gaussian noise is provided. It has already been shown that min I(x; e) is robust against covariate shift. Given the results shown above, it was validated that both min I(x; e) and min H(e) are robust to not only the covariate shift, but also the non-Gaussian noise. Here, two insights are provided on the robustness of min H(e) over the MSE criterion min E(e²) against non-Gaussian noises, in which E denotes the expectation. First, it has been suggested that min E(e²) is equivalent to minimizing the error entropy plus a KL divergence. Specifically, the following relationship applies:

min E(e²)⇔min H(e)+D_{KL}(p(e)∥φ(e)), (9)

where p(e) is the probability of error, φ(e) denotes a zero-mean Gaussian distribution. As the KL divergence is always non-negative, minimizing the MSE is equivalent to minimizing an upper bound of the error entropy. Eq. (9) also explains (partially) why in linear and Gaussian cases, the MSE and MEE are equivalent. Nevertheless, in case the error or noise follows a highly non-Gaussian distribution (especially when the signal-to-noise (SNR) value decreases), the MSE solution deviates from the MEE result, but the latter takes full advantage of high-order information of the error.

On the other hand, given the MSE criterion E(e²), the error entropy satisfies the following relationship:

$$H ( e ) \leq {max}_{E ( {ζ}^{2} ) = E ( {e}^{2} )} H ( ζ ) = \frac{1}{2} + \frac{1}{2} log 2 π + \frac{1}{2} log ( E ( {e}^{2} ) ) , \qquad ( 10 )$$

where ζ denotes a random variable whose second moment equals the MSE criterion E(e²).

This implies that the MSE criterion can be recognized as a robust MEE criterion in the minimax sense, because the following relationship applies:

$$\begin{matrix}{f}_{M S E}^{*} = {arg min}_{f ∈ F} E ( {e}^{2} ) \\ = {arg min}_{f ∈ F} \frac{1}{2} + \frac{1}{2} log 2 π + log ( E ( {e}^{2} ) ) \\ = {arg min}_{f ∈ F} {max}_{E ( {ζ}^{2} ) = E ( {e}^{2} )} H ( ζ ) ,\end{matrix} \qquad ( 11 )$$

where ƒ_{MSE}* denotes the solution with MSE criterion, [symbol] stands for the collection of all Borel measurable functions. Eq. (11) suggests that minimizing the MSE is equivalent to minimizing an upper bound of the error entropy.

Accordingly, the equivalence between min I(x; e) and the MEE criterion min H(e) has been established herein. Also, embodiments of the present invention showing the non-parameter independence measure based on the eigenspectrum of normalized Gram matrix of the projected data in the reproducing kernel Hilbert space were also discussed. It was shown that the independence measure according to embodiments of the present invention is more powerful than HSIC and other notable dependence measures. Moreover, the automatically differentiable property of the independence measure according to embodiments of the present invention can be a good surrogate to HSIC and other loss functions to train neural networks against changes in the input distribution or label noises. Another embodiment of the present invention could apply the independence measure as an information-theoretic cost function for robust machine learning. One example is the augmented space linear model, in which it is feasible to take full advantage of the joint space between input x and desired signal y (as an alternative to the joint space of x and e) to design learning systems. It is expected there will be more practical usage of both criteria (min I(x; e) and the MEE criterion min H(e)) in other machine learning applications.

For completeness, the mathematical formulations of different dependence measures that have been compared to the approach of embodiments of the present invention above are provided below. These other dependence measures include the KCCA, HSIC, dCov and QMI_CS.

The KCCA is defined as:

$${I}_{K C C A} ( x ; y ) = {sup}_{g ∈ {ℱ}^{1} , f ∈ {ℱ}^{2}} \frac{cov [ g ( x ) , f ( y ) ]}{\sqrt{var [ g ( x ) ] + κ {‖ g ‖}_{{ℱ}^{1}}^{2}} \sqrt{var [ f ( y ) ] + κ {‖ f ‖}_{{ℱ}^{2}}^{2}}} . \qquad ( 12 )$$

Therefore, KCCA can be interpreted as the supremum correlation of x and y over two “rich enough” reproducing kernel Hilbert spaces (RKHSs) [symbol] .

The HSIC is defined in accordance with the following: Given two separable [symbol] ¹ and [symbol] ² with associated feature maps ϕ₁ and ϕ₂, let the corresponding cross-covariance operator be:

C_{y}_{¹}_{,y}_{²}=E([ϕ₁(y¹)−μ₁)]⊗[ϕ₂(y²))−μ₂)])) (13)

where ⊗ represents the tensor product and HSIC is defined as the Hilbert-Schmidt norm of the cross-covariance operator as follows:

I_{HSIC}(y¹,y²)=∥C_{y}_{¹}_{y}_{²}∥_{HS} (14)

The dCov is defined in accordance with the following: Two random variables are independent if and only if their joint characteristic function can be factorized. This is the guiding principle behind the definition of distance covariance. In particular, given y¹∈ [symbol] ^{d}^{₁}, y²∈ [symbol] ^{d}^{₂} random variables (M=2), let ϕⱼ(ϕ₁₂) stand for the characteristic function of y₁ ([Yᵢ; y₂]) as follows:

ϕ₁₂(u¹,u²)= [symbol] [e^{i(u}^{¹}^{,y}^{¹}^{)+(u}^{²}^{,y}^{²}⁾] (15)

ϕⱼ(u^{j})= [symbol] [e^{i(u}^{^{j}}^{,y}^{^{j}}⁾], (j=1,2) (16)

where i=\sqrt{−1}, [symbol] ⋅ [symbol] the standard Euclidean inner product, and the [symbol] stands for expectation. The distance covariance is simply the L_{w}² norm of ϕ₁₂ and ϕ₁ϕ₂:

$${I}_{d C o v} ( {y}^{1} , {y}^{2} ) = {‖ {ϕ}_{12} - {ϕ}_{1} {ϕ}_{2} ‖}_{{L}_{w}^{2}} = {\int }_{{ℝ}^{{d}_{1} + {d}_{2}}} \sqrt{\vert {ϕ}_{12} ( {u}^{1} , {u}^{2} ) - {ϕ}_{1} ( {u}^{1} ) {ϕ}_{2} ( {u}^{2} ) \vert w ( {u}^{1} , {u}^{2} ) d {u}^{1} d {u}^{2}} \qquad ( 17 )$$

and the weight function w is defined as follows:

$$w ( {u}^{1} , {u}^{2} ) = \frac{1}{c ( {d}_{1} , α ) {{c ( {d}_{2} , α ) [ {‖ {u}^{1} ‖}_{2} ]}^{{d}_{1 +} α} [ {‖ {u}^{2} ‖}_{2} ]}^{{d}_{2 +} α}} \qquad ( 18 )$$

where α∈(0,2) and c(d,α) is defined as follows:

$$c ( d , α ) = \frac{2 π Γ ( 1 - \frac{α}{2} )}{α {2}^{α} Γ ( \frac{d + α}{2} )} \qquad ( 19 )$$

The QMI_CS is defined as:

$${I}_{{QMI}_{c s}} = log \frac{\begin{matrix}( {\int }_{{ℝ}^{{d}_{1}}} {\int }_{{ℝ}^{{d}_{2}}} {[ f ( {u}^{1} , {u}^{2} ) ]}^{2} {d}_{u 1} {d}_{u 2} ) \\ ( {\int }_{{ℝ}^{{d}_{1}}} {\int }_{{ℝ}^{{d}_{2} ,}} {{[ f ( {u}^{1} ) ]}^{2} [ f ( {u}^{2} ) ]}^{2} {d}_{u 1} {d}_{u 2} )\end{matrix}}{{[ {\int }_{{ℝ}^{{d}_{1}}} {\int }_{{ℝ}^{{d}_{2}}} f ( {u}^{1} , {u}^{2} ) f ( {u}^{1} ) f ( {u}^{2} ) {d}_{u 1} {d}_{u 2} ]}^{2}} \qquad ( 20 )$$

In the following, further embodiments of the present invention will be described with further details of the algorithms used for performing the respective functions. It will be understood by those skilled in the art that the following description also applies to the embodiments of the present invention described above even if different terminology or symbols are used.

As already discussed above, measuring the degree of dependence among multiple random variables plays a central role in machine learning, information theory, and statistics. In the following discussion, a unified view to existing information-theoretic multivariate dependence measures is first presented, aiming at illuminating their inner connections. The main idea of these measures is then generalized into a higher-level perspective by the Shearer's inequality. Motivated by this generalization according to an embodiment of the present invention, a pair of measures are provided, namely the normalized total correlation and the normalized dual total correlation, to quantify the multivariate dependence. Using the matrix-based Renyi's α-entropy functional estimator, it is demonstrated that both measures can be simply evaluated regardless of variable dimension, which enables a straightforward extension of the technique to random vectors of arbitrary dimension. Experiments validate the effectiveness of the measures according to embodiments of the present invention.

For linear dependence, measures such as the Pearson correlation coefficient have been widely used. For the more general case where the two variables contain non-linear dependence, the most well-known dependence measure is the Shannon mutual information, which has found a tremendous list of applications, including causality analysis, feature selection, image registration, and independent component analysis. However, real-world data often contains three or more variables which can exhibit multivariate (higher-order) dependencies. If bivariate based measures are used to identify multivariate dependence, spurious conclusions may happen. A vivid example is the XOR problem, in which Y=X₁⊕X₂, with X₁, X₂ being binary random processes with equal probability for all values of X₁ and X₂. Although I(X₁; Y)=I(X₂; Y)=0, the full dependency is synergistically contained between {X₁, X₂} and Y.

A desirable multivariate dependence measure I defined for X={X₁, X₂, . . . , X_{d}}∈ [symbol] ^{d} are expected to have the following properties: 1) dependence I(X₁, X₂, . . . , X_{d}) is invariant to permutation; 2) 0≤1(X)≤1, and I(X)=0 iff {X₁, X₂, . . . , X_{d}} are independent variables, I(X)=1 for a set of variables having strong functional relationship; 3) I(X₁, X₂, . . . , X_{d}) is invariant to strictly increasing transformation of Xᵢ variables. Over the last few years, numerous measures of multivariate dependence measures have been proposed. Notable examples include the multivariate maximal correlation (MAC), the Copula-based Kernel Dependency Measures (CKDM), the cumulative mutual information (CMI), and the Intrinsic Dimensional Dependency (IDD).

Despite substantial efforts having been made so far, the problem of measuring multivariate dependence (especially in nonparametric situations) still remains technically challenging, and researchers and practitioners are still not satisfied with the available measures and estimators. This is not difficult to understand as many of the aforementioned dependence measures are defined as some functionals of the density, thus an obvious way for their estimation requires to estimate the underlying densities at first. The density estimation, however, is notoriously difficult in high-dimensional space. On the other hand, the inner connections and theoretical foundations of existing information-theoretic multivariate dependence measures still remain unclear.

Contributions, advantages and improvements provided by embodiments of the present invention include:
providing a unified view of existing information-theoretic multivariate dependence measures and illustrating their inner connections.
Generalize the main idea of these measures into a higher-level perspective by the Shearer's inequality.
Based on the generalization, providing a pair of measures, namely the normalized total correlation (NTC) and the normalized dual total correlation (NDTC), to measure the multivariate dependence.
Using the matrix-based Rényi's α-entropy functional estimator, whereby the measures can be simply evaluated without estimating the underlying density functions.

It is demonstrated that the estimator according to embodiments of the present invention is less restricted to variable dimension, which enables a straightforward extension of the technique to random vectors. It is also empirically demonstrated that the measures according to embodiments of the present invention are able to quantify the amount of dependence among multiple random variables (or vectors). Moreover, it is also demonstrated that the measures according to embodiments of the present invention achieve improved technical performance against other state-of-the-art (SOTA) ones in terms of power and interpretability based on experiments with both synthetic and real-world data.

With respect to the problem formulation, given a random vector Y of dimension d that is characterized by N realizations, i.e., {yᵢ=[y₁ⁱ, y₂ⁱ, . . . , y_{d}ⁱ]^{T}}ᵢ₌₁^{N}, where the superscript denotes the sample index, and the subscript denotes dimension index, divide dimension index set {1, 2, . . . , d} into L (L≤d) non-overlapping and non-empty groups {G₁, G₂, . . . , G_{L}}, i.e., Uₗ₌₁^{L}Gₗ={1, 2, . . . , d} and (∀i≠j), Gᵢ∩Gⱼ=∅. Suppose Y(Gᵢ) denote the sub-vector of Y containing all dimension coordinates in Gᵢ, a statistic is developed that is able to quantify the complex (and possibly nonlinear) interactions between the L (disjoint) sub-vectors Y(G₁),Y(G₂), . . . , Y(G_{L}) from N observations. It can be provided that the derived statistic is strictly between 0 and 1 for improved interpretability. Meanwhile, the statistic is powerful to support a test of independence with the following null and alternative hypotheses:

H₀:Pr(Y(G₁),Y(G₂), . . . ,Y(G_{L}))=Pr(Y(G₁))Pr(Y(G₂)) . . . Pr(Y(G_{L}))

H₁:Pr(Y(Gᵢ),Y(G₂), . . . ,Y(G_{L}))≠Pr(Y(G₁))Pr(Y(G₂)) . . . Pr(Y(G_{L})),

where Pr refers to the probability distribution.

Despite tremendous efforts having been made, existing literature primarily focuses on two special scenarios: 1) the dependence associated with each dimension of a random vector (i.e., L=d), such as the MAC and the CKDM; and 2) the dependence between two random vectors (i.e., L=2), such as the RV coefficient and the distance covariance (dCov) coefficient. With respect to 1) multivariate correlation analysis, the total correlation (TC), formally defined as the Kullback-Leibler (KL) divergence from the joint distribution to the product of each marginal distributions, has become the workhorse of existing information-theoretic multivariate correlative analysis. Most of the recently proposed dependence measures, such as the MAC and universal dependency score (UDS), target functional relationships and compute their respective measures using Shannon discrete entropy that requires proper data discretization on continuous variables. It is illustrated below the inner connections between these measures. With respect to 2) random vector associations, common measures of linear dependence, such as the RV co-efficient and canonical correlation analysis (CCA), are computationally efficient and theoretically well understood, but consider only a limited class of association patterns, like monotonically increasing functions. Interestingly, despite the obvious deficiency, CCA has received increasing attention in either understanding the dynamics of learning of deep neural networks or quantifying the similarity on layer representations in deep transfer learning.

The development of non-linear dependence measures is challenging because of the radically larger amount of possible association patterns. Early efforts characterize Y(G₁) and Y(G₂) by two complete graphs where each observation is a node (N nodes in total) and the N(N−1)/2 edges are weighted by a dissimilarity measure. Then, two spanning subgraphs, usually k nearest-neighbor (kNN) graph, were built and a dependence measure was based on the number of edges common to the two graphs. Other well-known non-linear statistical dependence measures include the dCov, the Hilbert-Schmidt Independence Criterion (HSIC), and the Randomized Dependence Coefficient (RDC). RDC measures the dependence of Y(G₁) and Y(G₂) as the largest canonical correlation between k randomly chosen nonlinear projections of their copula transformations.

In the following, a unified view of information-theoretic dependency measures is provided. From a probability perspective, a multivariate dependency measure M that quantifies how much a random vector X={X₁, X₂, . . . , X_{d}}∈ [symbol] ^{d} deviates from statistical independence in each dimension can be simply represented as:

$$M ( X ) = diff ( p ( {X}_{1} , {X}_{2} , … , {X}_{d} ) \text{:} {\prod }_{i = 1}^{d} p ( {X}_{i} ) ) , \qquad ( 21 )$$

where diff refers to a measure of difference such as divergence or distance.

If one instantiates diff( ) with KL divergence, Eq. (21) reduces to the well-known Total Correlation (TC):

$$\begin{matrix}TC ( X ) = {D}_{K L} ( p ( {X}_{1} , {X}_{2} , … , {X}_{d} ) \vert \vert {\prod }_{i = 1}^{d} p ( {X}_{i} ) ) , & ( 22 ) \\ = [ {\sum }_{i = 1}^{d} H ( {X}_{i} ) ] - H ( {X}_{1} , {X}_{2} , … , {X}_{d} ) . & \end{matrix}$$

TC can be decomposed as:

$$TC ( {X}_{1} , {X}_{2} , … , {X}_{d} ) = {\sum }_{i = 1}^{d} I ( {X}_{i} ; {X}_{[ i - 1 ]} ) , \\ = {\sum }_{i = 1}^{d} H ( {X}_{i} ) - H ( {X}_{i} \vert {X}_{[ i - 1 ]} ) . \qquad ( 23 )$$

where, above or in the following, [n]:={1, 2, . . . , n} is abbreviated as [n]\{i}:=[n]\i.

Although this kind of progressive aggregation mechanism from small variable sets to large helps the measure scale well to high dimensionality, it is sensitive to the ordering of the variables, i.e., Eq. (23) is not permutation invariant. Let [symbol] _{d} be the set of bijective functions σ: {1, . . . , d}→{1, . . . , d}, a common strategy, motivated by the maximal correlation analysis approach, is to find an optimal permutation of {1, . . . , d} that maximizes TC:

$$M ( X ) = {max}_{σ ∈ {ℱ}_{d}} {\sum }_{i = 1}^{d} [ H ( {X}_{σ ( i )} ) - H ( {X}_{σ ( i )} \vert {X}_{σ ( 1 )} , {X}_{σ ( 2 )} , … , {X}_{σ ( i - 1 )} ) ] . \qquad ( 24 )$$

Most of existing information-theoretic multivariate dependency measures are developed along this line of research. Specifically, some measures directly estimate Eq. (22) with Shannon discrete entropy, which requires proper data discretization. To avoid estimation issues in high-dimensional space and improper data discretization when dealing with continuous variables, a general trend is to take advantage of the decomposition form of TC (i.e., Eq. (24)) and replace H(Xᵢ) and H(Xᵢ|X_{[i-1]}) with respectively the cumulative entropy (CE) and the conditional cumulative entropy (CCE). Notable examples include the MAC, the CMI, and the UMC. Compared with Shannon differential entropy, the cumulative entropy functional enjoys more desirable properties. For example, the conditional cumulative entropy of the variable X given Y is zero, if and only if X is a function of Y, but the vanishing of the conditional differential entropy of X given Y does not imply that X is a function of Y. However, one should note that, there are a total of d! possible permutations on the ordering of variables and a brute-force searching is impractical. As a result, different heuristic rules have been applied which always lead to sub-optimal performance.

Different from these efforts, the recently proposed CKDM instantiates diff( ) in Eq. (21) with the Maximum Mean Discrepancy (MMD) and measure the discrepancy between Pr(X₁, X₂, . . . , X_{d}) and Πᵢ₌₁^{d}Pr(Xᵢ) by first taking a copular transform on both distributions. Note that, a copula on Πᵢ₌₁^{d}Pr(Xᵢ) is simply a multivariate uniform distribution [0 1]^{d} if Pr(Xᵢ) is invertible. Although CKDM is theoretical sound and permutation invariant, the value of CKDM is not upper bounded, which makes it suffers from poor interpretability.

It is noted that TC is not the only non-negative measures of multivariate dependence. On the contrary, it can be seen as one simplest member of a large family, all obtained as special cases of an inequality due to Shearer. Given a set of d random variables X₁, X₂, . . . , X_{d}, denote φ the family of all subsets of [d] with the property that every member of [d] lies in at least k members of φ, the Shearer's inequality states that:

$$H ( {X}_{1} , {X}_{2} , … , {X}_{d} ) \leq \frac{1}{k} {\sum }_{S ∈ φ} H ( {X}_{i} , i ∈ S ) . \qquad ( 25 )$$

where TC is obtained when

$$φ = ( \begin{matrix}d \\ 1\end{matrix} ) .$$

Another important inequality arises when taking

$$φ = ( \begin{matrix}d \\ d - 1\end{matrix} ) ,$$

in which the Shearer's inequality suggests an alternative non-negative multivariate dependence measure as:

$$DTC ( X ) = [ {\sum }_{i = 1}^{d} H ( {X}_{[ d ] ∖ i} ) ] - ( d - 1 ) H ( {X}_{1} , {X}_{2} , … , {X}_{d} ) \qquad ( 26 )$$

Eq. (26) is also called the dual total correlation (DTC) and has an equivalent form as follows:

$$DTC = H ( {X}_{1} , {X}_{2} , … , {X}_{d} ) - [ {\sum }_{i = 1}^{d} H ( {X}_{i} \vert {X}_{[ d ] ∖ i} ) ] \qquad ( 27 )$$

This is because:

$$DTC ( X ) = [ {\sum }_{i = 1}^{d} H ( {X}_{[ d ] ∖ i} ) ] - ( d - 1 ) H ( {X}_{1} , {X}_{2} , … , {X}_{d} ) , \\ = H ( {X}_{1} , {X}_{2} , … , {X}_{d} ) - [ {\sum }_{i = 1}^{d} H ( {X}_{[ d ] ∖ i} ) - H ( {X}_{1} , {X}_{2} , … , {X}_{d} ) ] , \\ = H ( {X}_{1} , {X}_{2} , … , {X}_{d} ) - [ {\sum }_{i = 1}^{d} H ( {X}_{i} \vert {X}_{[ d ] ∖ i} ) ] . \qquad ( 28 )$$

In one embodiment of the present invention discussed in the following, DTC is implemented with Eq. (26) for simplicity. The Shearer's inequality has been recently refined further. From any of these inequalities, one can define a new notion of multivariate dependence by considering the gap between the two sides.

As mentioned above, TC and DTC are two special cases of the general multivariate dependence measure defined by the Shearer's inequality. To make TC and DTC more interpretable, i.e., taking values in the interval of [0 1], both measures are normalized as shown in Eq. (29) and Eq. (30):

$$\begin{matrix}NTC ( X ) = \frac{[ {\sum }_{i = 1}^{d} H ( {X}_{i} ) ] - H ( {X}_{1} , {X}_{2} , … , {X}_{d} )}{[ {\sum }_{i = 1}^{d} H ( {X}_{i} ) ] - {max}_{i} H ( {X}_{i} )} & ( 29 ) \\ NDTC ( X ) = \frac{[ {\sum }_{i = 1}^{d} H ( {X}_{[ d ] ∖ i} ) ] - ( d - 1 ) H ( {X}_{1} , {X}_{2} , … , {X}_{d} )}{H ( {X}_{1} , {X}_{2} , … , {X}_{d} )} & ( 30\end{matrix}$$

In the problem formulation above, it is suggested to measure the dependence associated with Y(G₁), Y(G₂), . . . , Y(G_{L}) with the same expressions of NTC and NDTC, which can be done as follows:

$$\begin{matrix} NTC ( Y ) = \frac{[ {\sum }_{i = 1}^{L} H ( Y ( {G}_{i} ) ) ] - H ( Y ( {G}_{1} ) , Y ( {G}_{2} ) , … , Y ( {G}_{L} ) )}{[ {\sum }_{i = 1}^{L} H ( Y ( {G}_{i} ) ) ] - {max}_{i} H ( Y ( {G}_{i} ) )} & ( 31 ) \\ NDTC ( Y ) = \frac{\begin{matrix}[ {\sum }_{i = 1}^{L} H ( Y ( {G}_{1} ) , … , Y ( {G}_{i - 1} ) , Y ( {G}_{i + 1} ) , … , Y ( {G}_{L} ) ) ] - \\ ( L - 1 ) H ( Y ( {G}_{1} ) , Y ( {G}_{2} ) , … , Y ( {G}_{L} ) )\end{matrix}}{H ( Y ( {G}_{1} ) , Y ( {G}_{2} ) , … , Y ( {G}_{L} ) )} & ( 32 )\end{matrix}$$

where, different from Xᵢ|ᵢ₌₁^{d} in Eqs. (29) and (30), Y(Gᵢ)|ᵢ₌₁^{L} in Eqs. (31) and (32) are not required to be exactly scalar random variables. In other words, there are no restrictions on the dimension of Y(Gᵢ). In this sense, if L=2, Eqs. (31) and (32) reduce to random vector association immediately. Moreover, the H in Eqs. (31) and (32) are not necessarily the Shannon entropy. In the following, the estimator to Eqs. (31) and (32) according to embodiments of the present invention is discussed, and the properties and improvements of the measures coupled with the estimator according to embodiments of the present invention are illustrated.

As can be seen from Eqs. (31) and (32), the measures according to embodiments of the present invention consist of multiple joint entropies associated with different groups of observations. On the one hand, one is able to estimate those joint entropies with Shannon discrete entropy. However, selecting a proper data discretization is a tricky technical problem and an improper discretization strategy may lead to serious estimation error. One the other hand, if the Shannon differential entropy is used, an issue arises naturally on the precise estimation of the (joint) probability distribution functions (PDF). If the prior information on PDF is not available (which is common in data-driven science), the PDF estimation is notoriously difficult in high-dimensional space.

To avoid this technical problem, embodiments of the present invention advantageously provide to use the matrix-based Renyi's α-entropy function and its multivariate extension, which suggest similar quantities that resemble quantum Renyi's entropy in terms of the normalized eigenspectrum of the Hermitian matrix of the projected data in the reproducing kernel Hilbert space (RKHS), thus estimating the entropy and joint entropy directly from data without PDF estimation. For brevity, the following definition can be used:
Definition 4.1: Let κ: χ×χ [symbol] [symbol] be a real valued positive definite kernel that is also infinitely divisible [37]. Given X={x¹, x², . . . , xⁿ} and the Gram matrix K obtained from evaluating a positive definite kernel K on all pairs of exemplars, that is (K)ᵢⱼ=κ(xⁱ,x^{j}), a matrix-based analogue to Rényi's α-entropy for a normalized positive definite (NPD) matrix A of size n×n, such that tr(A)=1, can be given by the following functional:

$${S}_{α} ( A ) = \frac{1}{1 - α} {log}_{2} ( t r ( {A}^{α} ) ) = \frac{1}{1 - α} {log}_{2} [ {\sum }_{i = 1}^{n} {{λ}_{i} ( A )}^{α} ] , \\ where \\ {A}_{i j} = \frac{1}{n} \frac{{K}_{i j}}{\sqrt{{K}_{i i} {K}_{j j}}} and {λ}_{i} ( A ) denotes the i \text{-} th eigenvalue of A . \qquad ( 33 )$$

Definition 4.2: Given a collection of n samples {sᵢ=(x₁ⁱ, x₂ⁱ, . . . , x_{L}ⁱ)}ᵢ₌₁ⁿ, where the superscript i denotes the sample index, each sample contains L (L≥2) measurements x₁∈χ₁, x₂∈χ₂, . . . , x_{L}∈χ_{L} obtained from the same realization, and the positive definite kernels κᵢ: χ₁×χ₁ [symbol] [symbol] , κ₂: χ₂×χ₂ [symbol] [symbol] , . . . , κ_{L}: χ_{L}×χ_{L} [symbol] [symbol] , a matrix-based analogue to Rényi's α-order joint-entropy among L variables can be defined as:

$${S}_{α} ( {A}_{1} , {A}_{2} , … , {A}_{L} ) = {S}_{α} ( \frac{{A}_{1} ∘ {A}_{2} ∘ … ∘ {A}_{L}}{t r ( {A}_{1} ∘ {A}_{2} ∘ … ∘ {A}_{L} )} ) , \qquad ( 34 )$$

where (A₁)ᵢⱼ=κ₁(x₁ⁱ,x₁^{j}), (A₂)ᵢⱼ=κ₂(x₂ⁱ,x₂^{j}), . . . , (A_{L})ᵢⱼ=κₖ (x_{L}ⁱ,x_{L}^{j}), and ∘ denotes the Hadamard product.

Eqs. (33) and (34) provide a novel matrix-based Renyi's α-order entropy and joint entropy can be straightforwardly computed by just evaluating the necessary Gram matrices according to embodiments of the present invention. Moreover, the novel definition is less sensitive to variable dimension. Based on this novel definition, Eqs. (31) and (32) can be simply expressed as:

$$\begin{matrix}NT {C}_{α} ( {A}_{1} , {A}_{2} , … , {A}_{L} ) = \frac{[ {\sum }_{i = 1}^{L} {S}_{α} ( {A}_{i} ) ] - {S}_{α} ( \frac{{A}_{1} ∘ {A}_{2} ∘ … ∘ {A}_{L}}{t r ( {A}_{1} ∘ {A}_{2} ∘ … ∘ {A}_{L} )} )}{[ {\sum }_{i = 1}^{L} {S}_{α} ( {A}_{i} ) ] - {max}_{i} {S}_{α} ( {A}_{i} )} , & ( 35 ) \\ {NDTC}_{α} ( {A}_{1} , {A}_{2} , … , {A}_{L} ) = \frac{\begin{matrix}[ {\sum }_{i = 1}^{L} ( \frac{{A}_{1} ∘ … ∘ {A}_{i - 1} ∘ {A}_{i + 1} … ∘ {A}_{L}}{tr ( {A}_{1} ∘ … ∘ {A}_{i - 1} ∘ {A}_{i + 1} … ∘ {A}_{L} )} ) ] - \\ ( L - 1 ) {S}_{α} ( \frac{{A}_{1} ∘ {A}_{2} ∘ … ∘ {A}_{L}}{tr ( {A}_{1} ∘ {A}_{2} ∘ … ∘ {A}_{L} )} )\end{matrix}}{{S}_{α} ( \frac{{A}_{1} ∘ {A}_{2} ∘ … ∘ {A}_{L}}{tr ( {A}_{1} ∘ {A}_{2} ∘ … ∘ {A}_{L} )} )} & ( 36 )\end{matrix}$$

In the following, properties and experimental results of the measures according to embodiments of the present invention (NTC and NDTC) are provided.

With respect to the properties of the measures, the following theorems are applicable:
Theorem 4.1: 0≤NTC_{α}(A₁, A₂, . . . A_{L})≤1 and 0≤NDTC_{α}(A₁, A₂, . . . , A_{L})≤1.
Theorem 4.2: NTC_{α}(A₁, A₂, . . . , A_{L}) and NDTC_{α}(A₁, A₂, . . . , A_{L}) reduce to zero iff Y(G₁), Y(G₂), . . . , Y(G_{L}) are independent.

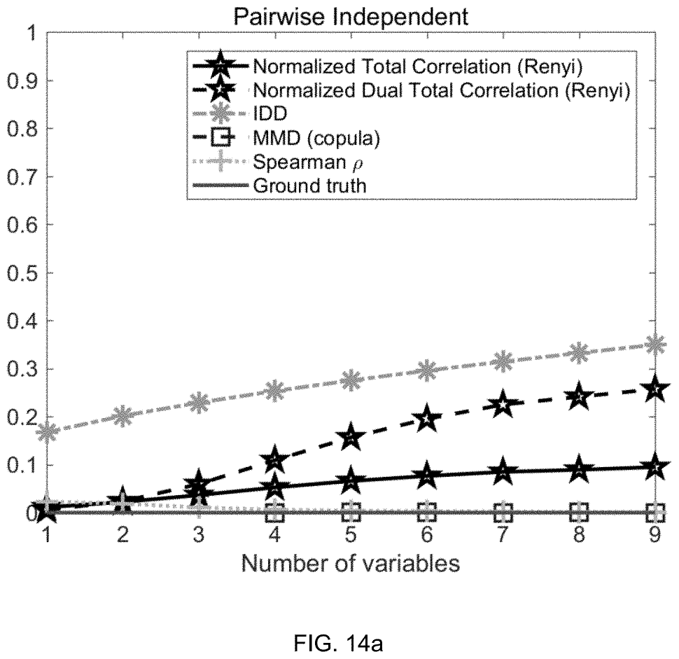

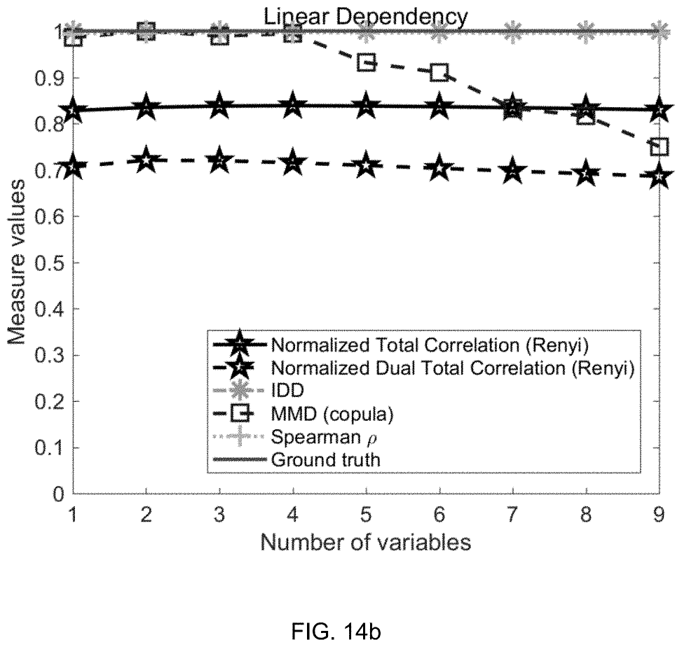

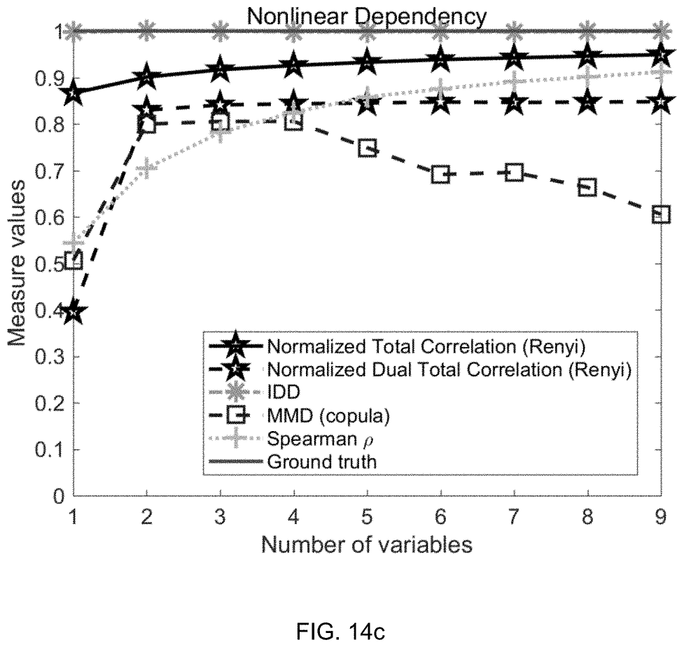

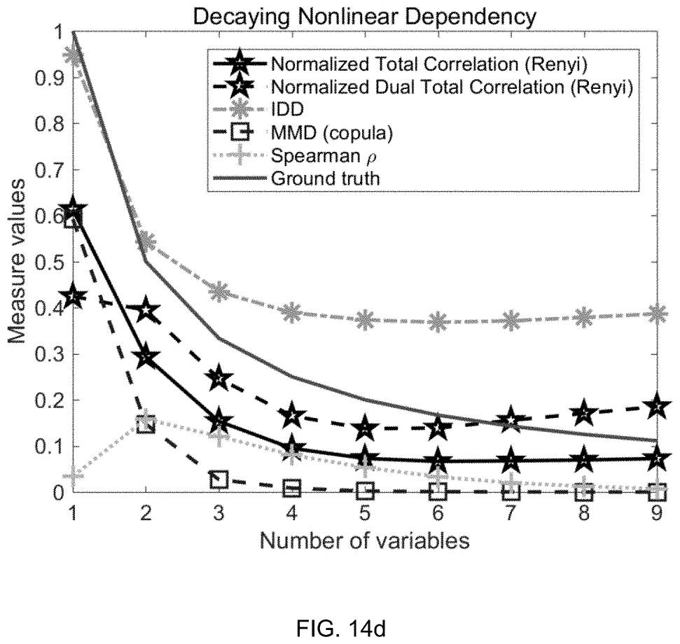

FIGS. 14a-14d show results of experiments on multivariate dependence measure using the measures according to embodiments of the present invention (Normalized Total Correlation (Renyi) and Normalized Dual Total Correlation (Renyi), and other dependence measures (IDD, MMD, Spearman), as well as the ground truth. Synthetic data was used and different correlations among each dimension of random vector X={X₁, X₂, . . . , X_{d}}∈ [symbol] ^{d} were crafted. The performance of the measures according to embodiments of the present invention (NTC_{α} and NDTC_{α}) is compared against three SOTA measures capable of handling multiple scalar variables, namely the multivariate extension of the Spearman's ρ, the CKDM (or MMD), and the IDD. Specifically, Spearman's ρ is a special association of concordance: if large (small) values of Xᵢ tend to be associated with large (small) values of Xⱼ, it is reflected in ρ. CKDM measures the MMD between Pr(X₁, X₂, . . . , X_{d}) and Πᵢ₌₁^{d}Pr(Xᵢ) after taking copular transform. IDD is able to identify variables that are embedded in low dimensional manifolds, thus having the potential to unveil dependence more than functional relationship. For all procedures, the hyper-parameters were set to their default values. FIGS. 14a-14d show the average value of the analyzed measures on the following relationships induced on d∈[1, 9] and n=1000 points:
Data for FIG. 14a: All dimensions of give data X are independent, which means that the total dependence of {X₁, X₂, . . . , X_{d}} should be zero.
Data for FIG. 14b: The first dimension X₁ is uniformly distributed in [0, 1], all other dimensions are the copy of the first dimension, i.e., Xᵢ=X₁ for i=2, 3, . . . , d. Therefore, the total dependence should be 1 (because {X₂, X₃, . . . , X_{d}} depends linearly only on X₁).
Data for FIG. 14c: The first dimension X₁ is uniformly distributed in [0, 1], and Xᵢ=(X₁)ⁱ for i=2, 3, . . . , d. Again, the total dependence should be 1 (because {X₂, X₃, . . . , X_{d}} depends nonlinearly only on X₁).
Data for FIG. 14d: There is a functional relationship between one dimension and the remaining dimensions:

$${X}_{1} = {( \frac{1}{d - 1} {\sum }_{i = 2}^{d} {X}_{i} )}^{2} ,$$

where {X₂, X₃, . . . , X_{d}} are uniformly and independently distributed. In this case, the strength of the overall dependence should decrease with the increase of dimension.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method for measuring dependence and independence of variables, the method comprising:
collecting data for the variables;
computing sample Gram matrices for the variables;
computing element-wise products of the sample Gram matrices; and
computing normalized mutual information of the variables using Renyi's α-order entropy function based on the element-wise products as an independence measure of the variables.

2. The method according to claim 1, wherein the independence measure is automatically differentiable.

3. The method according to claim 2, further comprising utilizing the independence measure as a loss function for training a neural network.

4. The method according to claim 1, wherein the variables are gene expressions, the method further comprising utilizing the independence measure to build a gene regulatory network (GRN) in which nodes represent levels of the gene expressions and edges represent interactions between genes.

5. The method according to claim 4, further comprising utilizing the GRN to design a compound, classify biological samples, design a treatment or to identify a most informative set of genes for a particular disease.

6. The method according to claim 1, wherein the variables are signals, the method further comprising utilizing the independence measure for blind source separation to distinguish different sources of the signals.

7. The method according to claim 6, wherein the blind source separation is used to distinguish between and listen to multiple speakers, or to measure propagation delay of seismic waves for petroleum exploration.

8. The method according to claim 1, wherein the independence measure between variable is between [0,1] with 0 indicating complete independence and 1 indicating direct functional dependence.

9. The method according to claim 1, wherein a normalization factor used to obtain the normalized mutual information is based on an upper bound of the mutual information using Shearer's inequality.

10. A system for measuring dependence and independence of variables comprising one or more processors which, alone or in combination, are configured to provide for execution of the following steps:
collecting data for the variables;
computing sample Gram matrices for the variables;
computing element-wise products of the sample Gram matrices; and
computing normalized mutual information of the variables using Renyi's α-order entropy function based on the element-wise products as an independence measure of the variables.

11. The system according to claim 10, wherein the independence measure is automatically differentiable, the system being further configured to utilize the independence measure as a loss function for training a neural network.

12. The system according to claim 10, wherein the variables are gene expressions, the system being further configured to utilize the independence measure to build a gene regulatory network (GRN) in which nodes represent levels of the gene expressions and edges represent interactions between genes, the GRN being useable to design a compound, classify biological samples, design a treatment or to identify a most informative set of genes for a particular disease.

13. The system according to claim 10, wherein the variables are signals, the system being further configured to utilize the independence measure for blind source separation to distinguish different sources of the source signals in order to distinguish between and listen to multiple speakers, or to measure propagation delay of seismic waves for petroleum exploration.

14. The system according to claim 10, wherein the independence measure between variables is between [0,1] with 0 indicating complete independence and 1 indicating direct functional dependence.

15. A tangible, non-transitory computer-readable medium having instructions thereon which, upon being executed by one or more processors, alone or in combination, provide for execution of the following steps:
collecting data for the variables;
computing sample Gram matrices for the variables;
computing element-wise products of the sample Gram matrices; and
computing normalized mutual information of the variables using Renyi's α-order entropy function based on the element-wise products as an independence measure of the variables.
